(19) United States
(12) Patent Application Publication
(10) Pub. No.: US 2018/0185404 A1
(43) Pub. Date: Jul. 5, 2018

(54) COMPOSITIONS AND METHODS FOR THE TREATMENT OF VIRAL INFECTION

(71) Applicant: Spring Bank Pharmaceuticals, Inc., Milford, MA (US)

(72) Inventors: Radhakrishnan P. IYER, Shrewsbury, MA (US); Seetharamaiyer PADMANABHAN, Lexington, MA (US)

(21) Appl. No.: 15/739,539

(22) PCT Filed: Jul. 1, 2016
(86) PCT No.: PCT/US16/40658

§ 371 Date: Dec. 22, 2017

Related U.S. Application Data
(60) Provisional application No. 62/188,300, filed on Jul. 2, 2015.

(51) Int. Cl. A61K 31/7084; A61K 31/7076; A61K 31/7072; A61P 31/16

(52) U.S. Cl. CPC A61K 31/7084; A61K 31/7076; A61K 2300/00; A61P 31/16; A61K 31/7072

(57) ABSTRACT

This invention relates to compositions and methods for the treatment of infection such as RSV, influenza, adenovirus, or rhinovirus.

### RELATED APPLICATIONS

This application claims priority to U.S. Provisional Application No. 62/188,300 filed Jul. 2, 2015, the contents of each of which are incorporated by reference in their entirety.

### FIELD OF INVENTION

This invention relates to compositions and methods for the treatment of a viral infection including respiratory infections such as those caused by viruses, for example, RSV, influenza, adenoviruses, and rhinoviruses.

### BACKGROUND OF INVENTION

There exists a significant unmet medical need for the treatment of existing and emerging viral diseases that pose a substantial health risk to millions of people worldwide. Despite the availability of vaccine, influenza A virus (flu) epidemics occur annually with 3-5 million severe cases in the young, elderly, and those with serious medical conditions, resulting in up to 500,000 deaths/year. Antiviral drugs such as oseltamivir, are only marginally effective and are ineffective in patients with flu complication. Human parainfluenza 3 (HPIV3) is one of the major respiratory RNA viruses that causes severe respiratory airway disease in infants and children and there is no vaccine for HPIV3. Respiratory syncytial virus (RSV)-related diseases affect infants, children, the immuno-compromised and elderly, causing an estimated 300,000 hospitalizations and 10,000 deaths yearly and is the principal driver of wheezing and asthma in children leading to higher medical costs. RSV and flu are a cause of acute asthma and COPD exacerbation in adults and accounts for 7.2% of adult asthma hospital admissions, 10.6% of pneumonia admissions, and 11.4% of COPD admissions. RSV is an important pathogen particularly in adults with chronic lung disease or impaired immunity. For severe RSV infections, prophylactic Palivizumab or Ribavirin are used but have significant side effects. Studies have shown that failure to activate an adequate innate immune response is primarily responsible for reduced virus clearance and exaggerated asthma and lung disease (i.e. massive pneumonia) and mortality. Overall therefore, effective treatments for flu, RSV and other respiratory infections remain a substantially unmet medical need. Rhinoviruses are the most common viral infectious agents in humans and are the predominant cause of the common cold. At least 99 serotypes of Human rhinoviruses affecting humans have been identified. There are no vaccines against these viruses as there is little-to-no cross-protection between serotypes.

### SUMMARY OF INVENTION

In one aspect, the present invention features a method for treating a subject infected with a viral infection such as respiratory syncytial virus (RSV), influenza, adenovirus or rhinovirus, the method comprising administering to the subject a compound of Formula (I), wherein the compound is selected from:

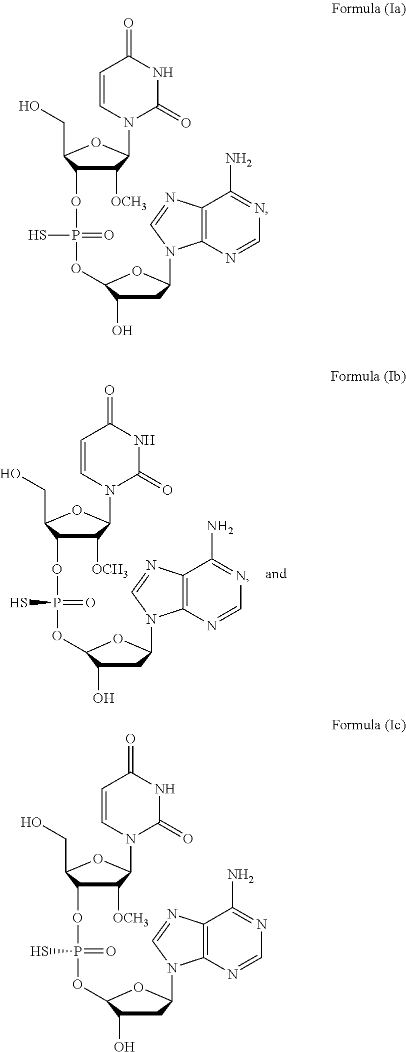

or a prodrug or pharmaceutically acceptable salt thereof. In an embodiment, the compound is administered through inhalation or by intratracheal delivery. In an embodiment, the compound is delivered orally or parenteraly. In an embodiment, the compound is delivered intranasally.

In an embodiment, the prodrug is a compound of Formula (P)

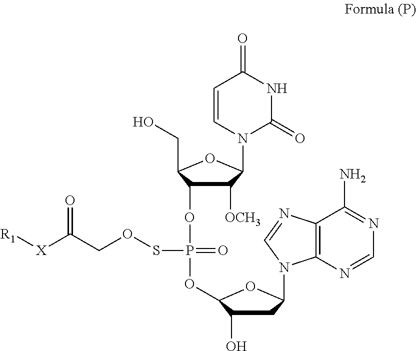

wherein,

X is a bond, O, NH, or S; and

R¹ is optionally substituted alkyl or heteroalkyl.

In an embodiment, X is a bond and R¹ is alkyl (e.g., t-butyl). In an embodiment, X is a bond and R¹ is heteroalkyl (e.g., an optionally substituted heteroalkyl).

In an embodiment, X is O and R¹ is alkyl or heteroalkyl.

In some embodiments, the prodrug of Formula (I) is a compound of Formula (II), wherein the compound is selected from:

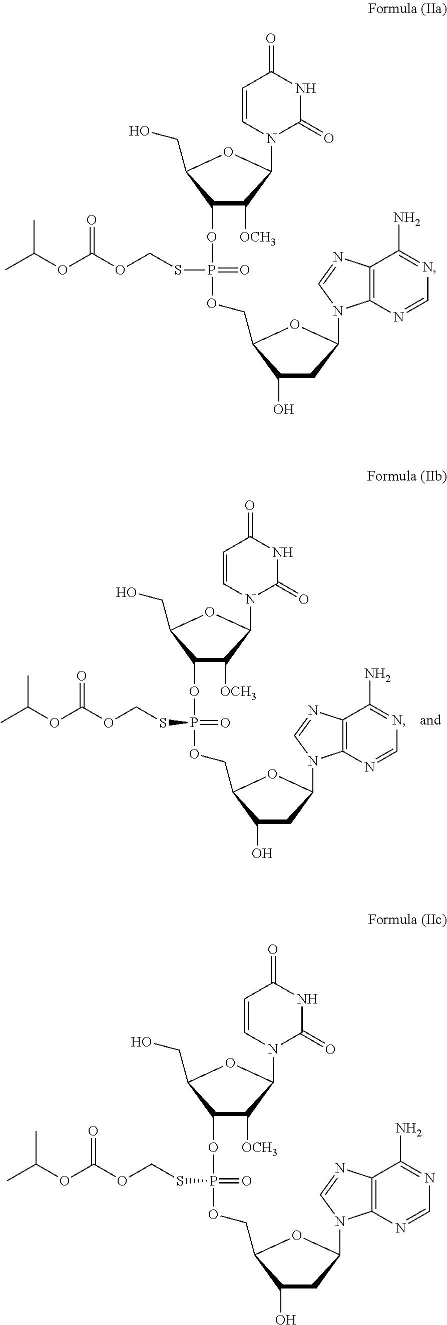

or a pharmaceutically acceptable salt thereof (e.g., a tartrate salt).

In an embodiment, the prodrug of Formula (I) is a compound of Formula (III), Formula (IV), Formula (V), or Formula (VI):

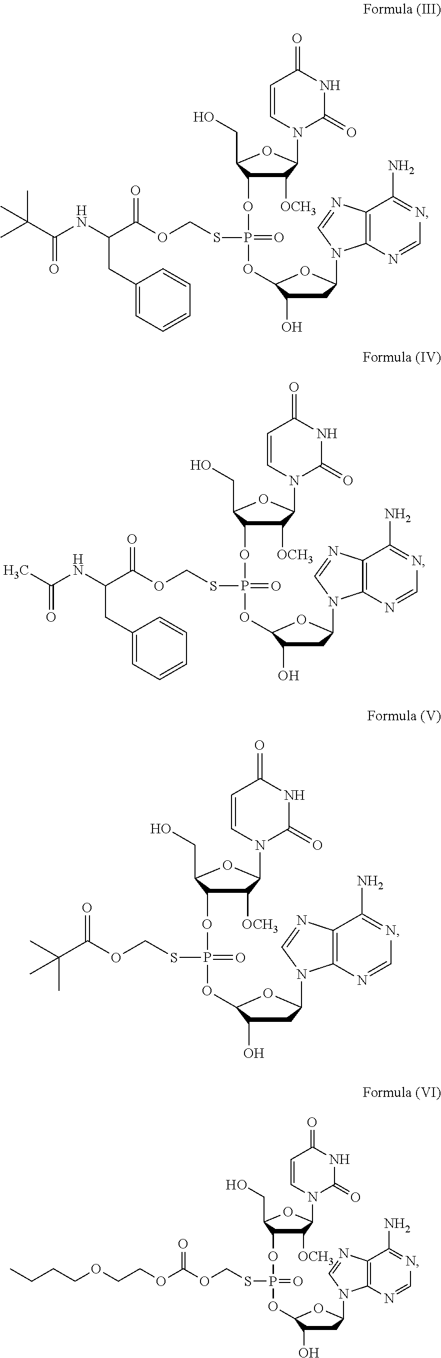

or a pharmaceutically acceptable salt thereof. In some embodiments, the individual isomeric forms of the prodrugs represented by Formulas III-VI may be employed, for example, as described in the ratios and percentages described below for compounds of Formula (I) and (II).

In some embodiments, the subject is administered a composition comprising a mixture of a compound described herein (e.g., compounds of Formula (I)). In some embodiments, the composition comprises a mixture of Formula (Ib) and Formula (Ic). In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater. In some embodiments, the mixture comprises a ratio of Formula (Ic) to Formula (Ib) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater.

In some embodiments, the composition comprises Formula (Ib) and comprises less than about 5% of Formula (Ic), e.g., less than about 4%, less than about 3%, less than about 2%, less than about 1%, less than about 0.5%, or less than about 0.1% of Formula (Ic), or is substantially free of Formula (Ic). In some embodiments, the composition comprises Formula (Ic) and comprises less than about 5% of Formula (Ib), e.g., less than about 4%, less than about 3%, less than about 2%, less than about 1%, less than about 0.5%, or less than about 0.1% of Formula (Ib), or is substantially free of Formula (Ib).

In some embodiments, the subject is administered a composition comprising a mixture of compounds of Formula (II). In some embodiments, the composition comprises a mixture of Formula (IIb) and Formula (IIc). In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater. In some embodiments, the mixture comprises a ratio of Formula (IIc) to Formula (IIb) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater.

In some embodiments, the composition comprises Formula (IIb) and comprises less than about 5% of Formula (IIc), e.g., less than about 4%, less than about 3%, less than about 2%, less than about 1%, less than about 0.5%, or less than about 0.1% of Formula (IIc), or is substantially free of Formula (IIc). In some embodiments, the composition comprises Formula (IIc) and comprises less than about 5% of Formula (IIb), e.g., less than about 4%, less than about 3%, less than about 2%, less than about 1%, less than about 0.5%, or less than about 0.1% of Formula (IIb), or is substantially free of Formula (IIb).

In some embodiments, the subject is a mammal. In some embodiments, the subject is a human. In some embodiments, the subject is an adult (e.g., over about 18 years of age, e.g., over about 65 years of age). In some embodiments, the subject is a child (e.g., under about 18 years of age, e.g., under about 5 years of age, e.g., under about 2 years of age). In some embodiments, the subject is immunocompromised (e.g., a subject that may have a weakened immune system relative to a reference standard, or may be suffering from an immune disease or condition).

In some embodiments, the method is a method of treating RSV. In some embodiments, the subject has been diagnosed with RSV infection. In some embodiments, the subject has been previously been infected with or diagnosed with RSV infection. In some embodiments, the genotype, serotype, subtype, or antigenic group of the RSV infection is known. In some embodiments, the RSV infection is RSV subtype A or RSV subtype B. In some embodiments, the subject is suffering from a severe RSV infection. In some embodiments, the subject is suffering from bronchiolitis, pneumonia, or other respiratory illness or condition.

In some embodiments, the subject is treatment naïve. In some embodiments, the subject has been previously treated for RSV infection (e.g., with an antibody or antiviral agent). In some embodiments, the subject has been previously treated for an immunodeficiency.

In some embodiments, the method is a method of treating influenza. In an embodiment, the subject has been diagnosed with influenza. In an embodiment the influenza is Type A, Type B, or Type C. In an embodiment, the method reduces the severity or prevents a complication of influenza (e.g., viral pneumonia, secondary bacterial pneumonia, sinus infections, and worsening of previous health problems such as asthma or heart failure).

In some embodiments, the method involves treating an infection that causes a common cold. In an embodiment, the subject has been diagnosed with a rhinovirus infection that causes a common cold.

In some embodiments, the method comprises daily administration of said dosage. In some embodiments, the administration is once daily. In some embodiments, the administration is greater than once daily, e.g., twice daily, three times daily, four times daily. In some embodiments, the method comprises administration of said dosage at a frequency less than once a day, e.g., once every 36 hours, once every other day, or once a week. In some embodiments, the method consists of treatment with the compounds prophylactically.

In some embodiments, the dosage comprises about 0.5 mg/kg to about 100 mg/kg. In some embodiments, the dosage comprises about 0.5 mg/kg to about 95 mg/kg, about 90 mg/kg, about 85 mg/kg, about 80 mg/kg, about 75 mg/kg, about 70 mg/kg, about 65 mg/kg about 60 mg/kg, about 55 mg/kg, about 50 mg/kg, about 45 mg/kg, about 40 mg/kg, about 35 mg/kg, about 30 mg/kg, about 25 mg/kg, about 20 mg/kg, about 15 mg/kg, or about 10 mg/kg. In some embodiments, the dosage comprises about 0.5 mg/kg to about 50 mg/kg. In some embodiments, the dosage comprises about 0.5 mg/kg to about 40 mg/kg.

In some embodiments, the dosage is greater than about 0.5 mg/kg, e.g., about 1.0 mg/kg, about 1.5 mg/kg, about 2 mg/kg, about 3 mg/kg, about 4 mg/kg, about 5 mg/kg, about 10 mg/kg about 15 mg/kg, about 20 mg/kg, about 25 mg/kg, about 30 mg/kg, about 35 mg/kg, about 40 mg/kg, about 50 mg/kg, about 55 mg/kg, about 60 mg/kg, about 65 mg/kg, about 70 mg/kg, about 75 mg/kg, about 80 mg/kg, about 85 mg/kg, or about 90 mg/kg up to about 100 mg/kg. In some embodiments, the dosage comprises about 5 mg/kg to about 50 mg/kg. In some embodiments, the dosage comprises about 10 mg/kg to about 50 mg/kg. In some embodiments, the dosage comprises about 15 mg/kg to about 50 mg/kg.

In an embodiment, the dosage is configured for inhalation or intratracheal administration. In some embodiments, the dosage is configured for intranasal administration. In some embodiments, the dosage is configured for oral or parenteral administration. In some embodiments, the dosage comprises a liquid or a solid dosage form. In some embodiments, the liquid dosage form comprises a suspension, a solution, an emulsion, a spray, a mist, an aerosol, an elixir, or a syrup. In some embodiments, the spray, mist, or aerosol mist comprises particles between about 0.01 microns to about 10 microns in size. In some embodiments, the spray, mist, or aerosol comprises particles of about 0.01 micron, about 0.025 micron, about 0.05 micron, about 0.075 micron, about 0.1 micron, about 0.25 micron, about 0.5 micron, about 0.75 micron, about 1 micron, about 1.25 microns, about 1.5 microns, about 1.75 microns, about 2 microns, about 3 microns, about 4 microns, about 5 microns, about 6 microns, about 7 microns, about 8 microns, about 9 microns, or about 10 microns in size, although the actual size of the particles may be outside of this range.

In some embodiments, the solid dosage form comprises tablets, capsules, soft gels, granules, particles, a powder, a gel, or a microencapsulated dosage form. In some embodiments, the granules, particles, powder, gel, or microencapsulated dosage form comprises particles between about 0.01 microns to about 10 microns in size. In some embodiments, the granules, particles, powder, gel, or microencapsulated dosage form comprises particles of about 0.01 micron, about 0.025 micron, about 0.05 micron, about 0.075 micron, about 0.1 micron, about 0.25 micron, about 0.5 micron, about 0.75 micron, about 1 micron, about 1.25 microns, about 1.5 microns, about 1.75 microns, about 2 microns, about 3 microns, about 4 microns, about 5 microns, about 6 microns, about 7 microns, about 8 microns, about 9 microns, or about 10 microns in size, although the actual size of the particles may be outside of this range.

In some embodiments, the compound is administered using a nebulizer or an inhaler (e.g., a dry powder inhaler, a metered dose inhaler).

In some embodiments, the compound is administered as a pharmaceutical composition. In some embodiments, the composition comprises a pharmaceutically acceptable excipient, carrier, or additive.

In some embodiments, the methods described herein further comprise analyzing or receiving analysis of a lung specimen or blood specimen from the subject. In some embodiments, the lung specimen or blood specimen is analyzed between about 4 hours and about 96 hours post-infection with RSV (e.g., between about 16 hours and about 72 hours). In some embodiments, the blood specimen is analyzed for viral load, IL6 levels, TNF levels, IFN-β levels, RSV nucleocapsid (N) protein levels, RIG-I levels, or NOD2 levels (e.g., by RT-PCR). In some embodiments, the lung specimen is analyzed for viral load, IL6 levels, TNF levels, IFN-β levels, RSV nucleocapsid (N) protein levels, RIG-I levels, or NOD2 levels (e.g., by RT-PCR, plaque assay, or histological staining). In some embodiments, the lung specimen or blood specimen is analyzed for the expression level of interferon (e.g., interferon alfa or interferon beta), an interferon stimulating protein (e.g., ISG15, CXCL10, OAS 1), or other cytokines. In some embodiments, the lung specimen is analyzed for obstruction, inflammation, infiltration of immune cells, size of alveolar spaces, and necrosis.

In some embodiments, the methods described herein further comprise the administration of a therapeutically effective amount of an additional agent. In some embodiments, the additional agent is an antiviral agent, antibacterial agent, an anticancer agent, anti-inflammatory agent, an antibody, a bronchodilator, an analgesic agent, an antipyretic agent, a cough suppressant, or an antihistamine. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the antiviral agent comprises a capsid inhibitor, an entry inhibitor, a secretion inhibitor, a microRNA, an antisense RNA agent, an RNAi agent, or other agent designed to inhibit viral RNA. In some embodiments, the nucleoside analog comprises entecavir, lamuvidine, adefovir, darunavir, sofosbuvir, telaprevir, tenofovir, zidovudine, ribavirin, lamivudine, entecavir, or AL-8176.

In some embodiments, the anticancer agent comprises methotrexate, 5-fluorouracil, doxorubicin, vincristine, bleomycin, vinblastine, dacarbazine, toposide, cisplatin, epirubicin, and sorafenib tosylate. In some embodiments, the anti-inflammatory agent, analgesic agent, or antipyretic agent comprises acetaminophen, aspirin, ibuprofen, naproxen, fenoprofen, dexibuprofen, or ketoprofen. In some embodiments, the bronchodilator comprises albuterol, salbutamol, epinephrine (e.g., racemic epinephrine), levosalbutamol, pirbuterol, ephedrine, terbutaline, salmeterol, clenbuterol, formoterol, bambuterol, or indacaterol.

In another aspect, the present invention features a method for preventing a viral infection such as respiratory syncytial virus (RSV), influenza, adenovirus or rhinovirus, the method comprising administering to the subject a compound of Formula (I), wherein the compound is selected from:

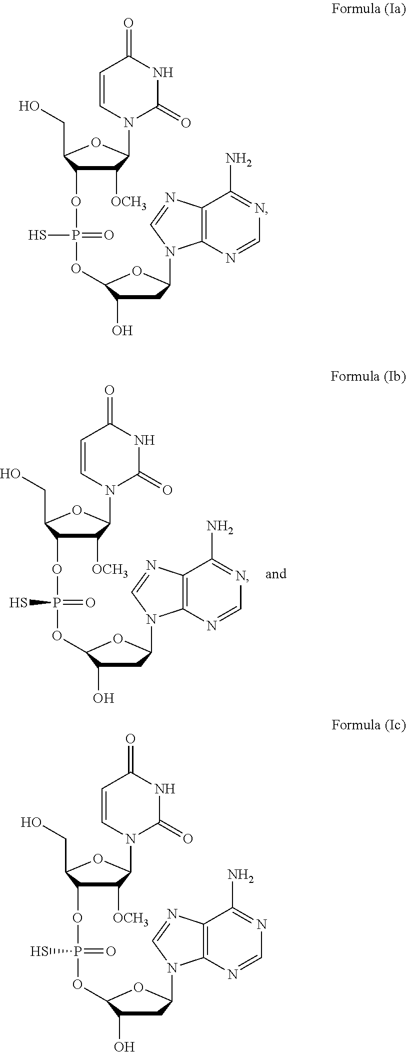

or a prodrug or pharmaceutically acceptable salt thereof. In an embodiment, the compound is administered through intranasal delivery, inhalation or by intratracheal delivery. In an embodiment, the compound is delivered orally or parenteraly.

In an embodiment, the prodrug is a compound of Formula (P)

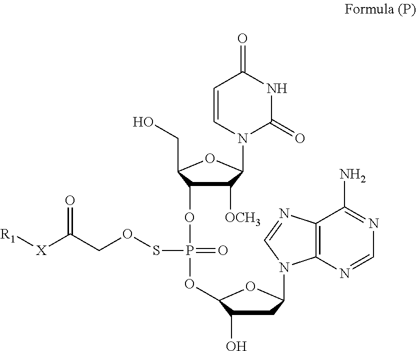

wherein,

X is a bond, O, NH, or S; and

R¹ is optionally substituted alkyl or heteroalkyl.

In an embodiment, X is a bond and R¹ is alkyl (e.g., t-butyl). In an embodiment, X is a bond and R¹ is heteroalkyl (e.g., an optionally substituted heteroalkyl).

In an embodiment, X is O and R¹ is alkyl or heteroalkyl.

In some embodiments, the prodrug of Formula (I) is a compound of Formula (II), wherein the compound is selected from:

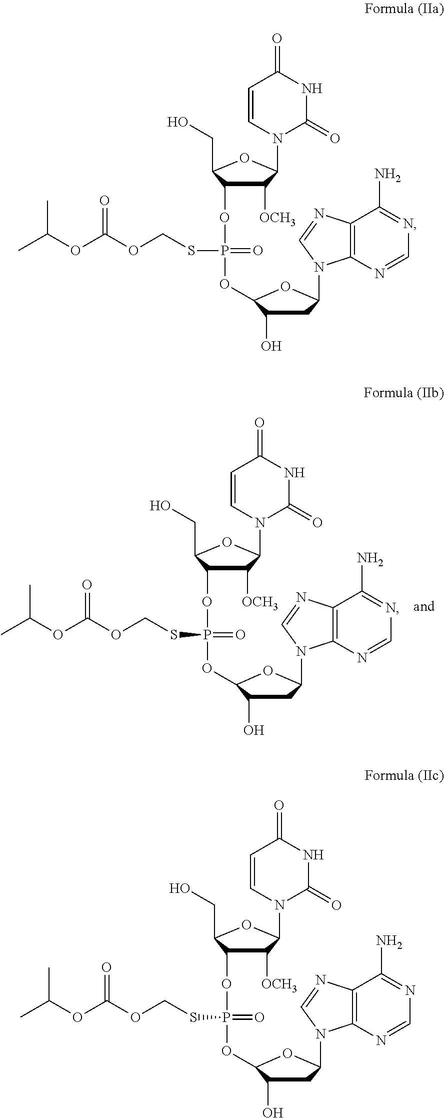

or a pharmaceutically acceptable salt thereof (e.g., a tartrate salt).

In an embodiment, the prodrug of Formula (I) is a compound of Formula (III), Formula (IV), Formula (V), or Formula (VI):

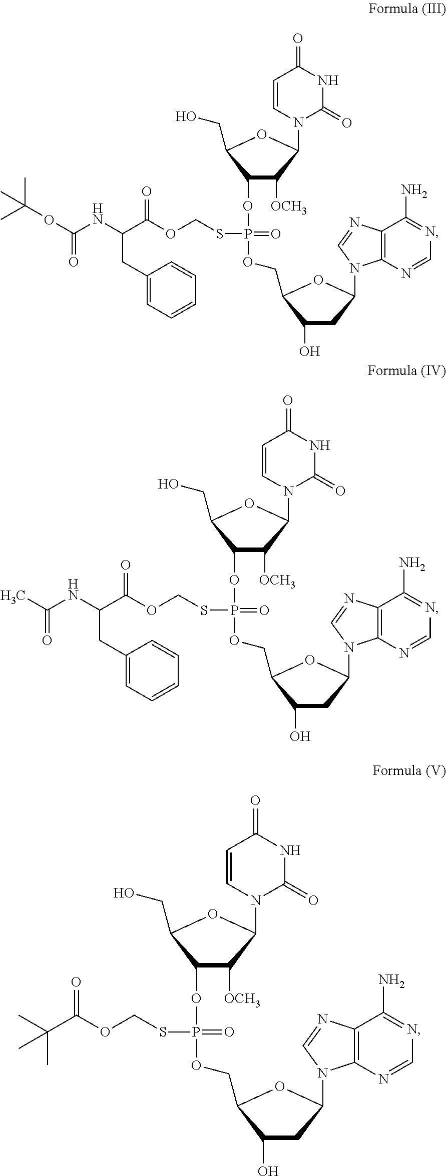

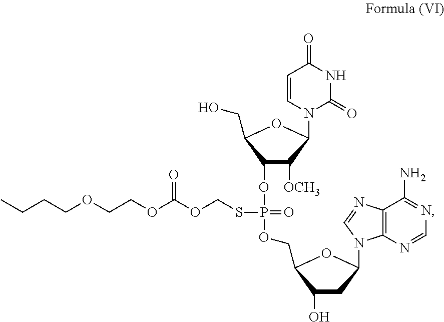

or a pharmaceutically acceptable salt thereof. In some embodiments, the individual isomeric forms of the prodrugs represented by Formulas III-VI may be employed, for example, as described in the ratios and percentages described below for compounds of Formula (I) and (II).

In some embodiments, the subject is administered a composition comprising a mixture of compounds of Formula (I). In some embodiments, the composition comprises a mixture of Formula (Ib) and Formula (Ic). In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater. In some embodiments, the mixture comprises a ratio of Formula (Ic) to Formula (Ib) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater.

In some embodiments, the composition comprises Formula (Ib) and comprises less than about 5% of Formula (Ic), e.g., less than about 4%, less than about 3%, less than about 2%, less than about 1%, less than about 0.5%, or less than about 0.1% of Formula (Ic), or is substantially free of Formula (Ic). In some embodiments, the composition comprises Formula (Ic) and comprises less than about 5% of Formula (Ib), e.g., less than about 4%, less than about 3%, less than about 2%, less than about 1%, less than about 0.5%, or less than about 0.1% of Formula (Ib), or is substantially free of Formula (Ib).

In some embodiments, the subject is administered a composition comprising a mixture of compounds of Formula (II). In some embodiments, the composition comprises a mixture of Formula (IIb) and Formula (IIc). In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater. In some embodiments, the mixture comprises a ratio of Formula (IIc) to Formula (IIb) of about 51:49, about 52: 48, about 53:47, about 54:46, about 55:45, about 60:40, about 65:35, about 70:30, about 75:25, about 80:20, about 85:15, about 90:10, about 95:5, or about 99:1 or greater.

In some embodiments, the composition comprises Formula (IIb) and comprises less than about 5% of Formula (IIc), e.g., less than about 4%, less than about 3%, less than about 2%, less than about 1%, less than about 0.5%, or less than about 0.1% of Formula (IIc), or is substantially free of Formula (IIc). In some embodiments, the composition comprises Formula (IIc) and comprises less than about 5% of Formula (IIb), e.g., less than about 4%, less than about 3%, less than about 2%, less than about 1%, less than about 0.5%, or less than about 0.1% of Formula (IIb), or is substantially free of Formula (IIb).

In some embodiments, the subject is a mammal. In some embodiments, the subject is a human. In some embodiments, the subject is an adult (e.g., over about 18 years of age, e.g., over about 65 years of age). In some embodiments, the subject is a child (e.g., under about 18 years of age, e.g., under about 5 years of age, e.g., under about 2 years of age). In some embodiments, the subject is immunocompromised (e.g., a subject that may have a weakened immune system relative to a reference standard, or may be suffering from an immune disease or condition).

In some embodiments, the method is a method of preventing RSV. In some embodiments, the subject has been previously been infected with or diagnosed with RSV infection. In some embodiments, the genotype, serotype, subtype, or antigenic group of the RSV infection is known. In some embodiments, the RSV infection is RSV subtype A or RSV subtype B. In some embodiments, the subject is suffering from a severe RSV infection. In some embodiments, the subject is suffering from bronchiolitis, pneumonia, or other respiratory illness or condition.

In some embodiments, the method is a method of preventing influenza. In an embodiment, the subject has been diagnosed with influenza. In an embodiment the influenza is Type A, Type B, or Type C. In an embodiment, the method reduces the severity or prevents a complication of influenza (e.g., viral pneumonia, secondary bacterial pneumonia, sinus infections, and worsening of previous health problems such as asthma or heart failure).

In some embodiments, the method is a method of preventing a common cold. In an embodiment, the subject has been diagnosed with rhinovirus infection.

In some embodiments, the subject is treatment naïve. In some embodiments, the subject has been previously treated for RSV infection (e.g., with an antibody or antiviral agent). In some embodiments, the subject has been previously treated for an immunodeficiency.

In some embodiments, the method comprises daily administration of said dosage. In some embodiments, the administration is once daily. In some embodiments, the administration is greater than once daily, e.g., twice daily, three times daily, four times daily. In some embodiments, the method comprises administration of said dosage at a frequency less than once a day, e.g., once every 36 hours, once every other day, or once a week.

In some embodiments, the dosage comprises about 0.5 mg/kg to about 100 mg/kg. In some embodiments, the dosage comprises about 0.5 mg/kg to about 95 mg/kg, about 90 mg/kg, about 85 mg/kg, about 80 mg/kg, about 75 mg/kg, about 70 mg/kg, about 65 mg/kg about 60 mg/kg, about 55 mg/kg, about 50 mg/kg, about 45 mg/kg, about 40 mg/kg, about 35 mg/kg, about 30 mg/kg, about 25 mg/kg, about 20 mg/kg, about 15 mg/kg, or about 10 mg/kg. In some embodiments, the dosage comprises about 0.5 mg/kg to about 50 mg/kg. In some embodiments, the dosage comprises about 0.5 mg/kg to about 40 mg/kg.

In some embodiments, the dosage is greater than about 0.5 mg/kg, e.g., about 1.0 mg/kg, about 1.5 mg/kg, about 2 mg/kg, about 3 mg/kg, about 4 mg/kg, about 5 mg/kg, about 10 mg/kg about 15 mg/kg, about 20 mg/kg, about 25 mg/kg, about 30 mg/kg, about 35 mg/kg, about 40 mg/kg, about 50 mg/kg, about 55 mg/kg, about 60 mg/kg, about 65 mg/kg, about 70 mg/kg, about 75 mg/kg, about 80 mg/kg, about 85 mg/kg, or about 90 mg/kg up to about 100 mg/kg. In some embodiments, the dosage comprises about 5 mg/kg to about 50 mg/kg. In some embodiments, the dosage comprises about 10 mg/kg to about 50 mg/kg. In some embodiments, the dosage comprises about 15 mg/kg to about 50 mg/kg.

In some embodiments, the dosage comprises a liquid or a solid dosage form. In some embodiments, the liquid dosage form comprises a suspension, a solution, an emulsion, a spray, a mist, an aerosol, an elixir, or a syrup. In some embodiments, the spray, mist, or aerosol mist comprises particles between about 0.01 microns to about 10 microns in size. In some embodiments, the spray, mist, or aerosol comprises particles of about 0.01 micron, about 0.025 micron, about 0.05 micron, about 0.075 micron, about 0.1 micron, about 0.25 micron, about 0.5 micron, about 0.75 micron, about 1 micron, about 1.25 microns, about 1.5 microns, about 1.75 microns, about 2 microns, about 3 microns, about 4 microns, about 5 microns, about 6 microns, about 7 microns, about 8 microns, about 9 microns, or about 10 microns in size, although the actual size of the particles may be outside of this range.

In some embodiments, the solid dosage form comprises tablets, capsules, soft gels, granules, particles, a powder, a gel, or a microencapsulated dosage form. In some embodiments, the granules, particles, powder, gel, or microencapsulated dosage form comprises particles between about 0.01 microns to about 10 microns in size. In some embodiments, the granules, particles, powder, gel, or microencapsulated dosage form comprises particles of about 0.01 micron, about 0.025 micron, about 0.05 micron, about 0.075 micron, about 0.1 micron, about 0.25 micron, about 0.5 micron, about 0.75 micron, about 1 micron, about 1.25 microns, about 1.5 microns, about 1.75 microns, about 2 microns, about 3 microns, about 4 microns, about 5 microns, about 6 microns, about 7 microns, about 8 microns, about 9 microns, or about 10 microns in size, although the actual size of the particles may be outside of this range.

In some embodiments, the compound is administered using a nebulizer or an inhaler (e.g., a dry powder inhaler, a metered dose inhaler).

In some embodiments, the compound is administered as a pharmaceutical composition. In some embodiments, the composition comprises a pharmaceutically acceptable excipient, carrier, or additive.

In some embodiments, the methods described herein further comprise analyzing or receiving analysis of a lung specimen or blood specimen from the subject. In some embodiments, the lung specimen or blood specimen is analyzed between about 4 hours and about 96 hours post-infection with RSV (e.g., between about 16 hours and about 72 hours). In some embodiments, the blood specimen is analyzed for viral load, IL6 levels, TNF levels, IFN-β levels, RSV nucleocapsid (N) protein levels, RIG-I levels, or NOD2 levels (e.g., by RT-PCR). In some embodiments, the lung specimen is analyzed for viral load, IL6 levels, TNF levels, IFN-β levels, RSV nucleocapsid (N) protein levels, RIG-I levels, or NOD2 levels (e.g., by RT-PCR, plaque assay, or histological staining). In some embodiments, the lung specimen or blood specimen is analyzed for the expression level of interferon (e.g., interferon alfa or interferon beta), an interferon stimulating protein (e.g., ISG15, CXCL10, OAS 1), or other cytokines. In some embodiments, the lung specimen is analyzed for obstruction, inflammation, infiltration of immune cells, size of alveolar spaces, and necrosis.

In some embodiments, the methods described herein further comprise the administration of a therapeutically effective amount of an additional agent. For example, in an embodiment, a compound described herein (e.g., a compound of Formula (I) or a prodrug thereof, can be used concomitantly with another agent (e.g., an agent to treat another viral infection such as HBV or HIV). For example, in an embodiment a subject having another viral infection such as HIV and is being treated with an HIV agent may develop another virus described herein such as RSV or influenza, and be treated with a compound of Formula (I) or a prodrug thereof. In an embodiment a subject having cancer and is being treated with an anti-cancer agent may develop a viral infection described herein such as RSV or influenza, and be treated with a compound of Formula (I) or a prodrug thereof. In some embodiments, the additional agent is an antiviral agent, antibacterial agent, an anticancer agent, anti-inflammatory agent, an antibody, a bronchodilator, an analgesic agent, an antipyretic agent, a cough suppressant, or an antihistamine. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the antiviral agent comprises a capsid inhibitor, an entry inhibitor, a secretion inhibitor, a microRNA, an antisense RNA agent, an RNAi agent, or other agent designed to inhibit viral RNA. In some embodiments, the nucleoside analog comprises entecavir, lamivudine, adefovir, darunavir, sofosbuvir, telaprevir, tenofovir, zidovudine, ribavirin, lamivudine, entecavir, or AL-8176.

In some embodiments, the anticancer agent comprises methotrexate, 5-fluorouracil, doxorubicin, vincristine, bleomycin, vinblastine, dacarbazine, toposide, cisplatin, epirubicin, and sorafenib tosylate. In some embodiments, the anti-inflammatory agent, analgesic agent, or antipyretic agent comprises acetaminophen, aspirin, ibuprofen, naproxen, fenoprofen, dexibuprofen, or ketoprofen. In some embodiments, the bronchodilator comprises albuterol, salbutamol, epinephrine (e.g., racemic epinephrine), levosalbutamol, pirbuterol, ephedrine, terbutaline, salmeterol, clenbuterol, formoterol, bambuterol, or indacaterol.

### BRIEF DESCRIPTION OF THE DRAWINGS

FIGS. 1A-1B are graphs depicting IL-6 production in the airways of RSV-infected mice. BALB/c mice (n=5 per group) were administered either water (vehicle) or Formula (II) (10 mg/kg) intratracheally. 16 hrs after administration, the mice were infected with RSV in the presence of either Formula (II) or water. At either 2 days or 4 days post-RSV infection, lungs were collected from the mice and the levels of IL-6 in the airways were determined through ELISA analysis of lung homogenate. Lungs of mice treated with the compound 1 show decreased levels of the pro-inflammatory cytokine 11-6.

FIGS. 2A-2B are graphs depicting TNF-alpha production in the airways of RSV-infected mice. BALB/c mice (n=5 per group) were administered either water (vehicle) or Formula (II) (10 mg/kg) intratracheally. 16 hrs after administration, the mice were infected with RSV in the presence of either Formula (II) or water. At either 2 days or 4 days post-RSV infection, lungs were collected from the mice and the levels of TNF in the airways were determined through ELISA analysis of lung homogenate. Lungs of mice treated with the compound 1 show decreased levels of the pro-inflammatory cytokine TNF-alpha.

FIG. 3 is a graph depicting IFN-β production in the airways of RSV-infected mice. Similar to the procedures outlined above, BALB/c mice (n=5 per group) were administered either water (vehicle) or Formula (II) (10 mg/kg) intratracheally. 16 hrs after administration, the mice were infected with RSV in the presence of either Formula (II) or water. Two days post-RSV infection, lungs were collected from the mice and the levels of IFN-β in the airways were determined through ELISA analysis of lung homogenate. Treatment with Compound 1 results in increased production of IFN initially which then tapers off following inhibition of viral replication and reduction in infection. Overall, this results in control of infection and reduction inflammatory response.

FIGS. 4A-4B are agarose gels showing reduced RSV infection in the airways of Formula (II)-treated mice. BALB/c mice (n=5 per group) were administered either water (vehicle) Formula (II) (10 mg/kg) intratracheally. 16 hrs after administration, the mice were infected with RSV in the presence of either Formula (II) or water. At either 2 days or 4 days post-RSV infection, lungs were collected and the extent of RSV infection was investigated through quantification of RSV nucleocapsid (N) gene expression by RT-PCR. GADPH expression served as a loading control. Each lane for RSV+water and RSV+SB represents the results from a single mouse, and the data is representative of three mice per group.

FIG. 5 depicts agarose gel analysis of RIG-I expression in the lung of RSV-infected mice. BALB/c mice (n=5 per group) were administered either water (vehicle) or Formula (II) (10 mg/kg) intratracheally. 16 hrs after administration, the mice were infected with RSV in the presence of either Formula (II) or water. At 2 days post-RSV infection, lungs were collected and the extent of RIG-I expression in the respiratory tract was examined by RT-PCR. GADPH expression served as a loading control. Each lane for RSV+water and RSV+SB represents the results from a single mouse, and the data is representative of three mice per group.

FIG. 6 depicts agarose gel analysis of NOD2 expression in the lung of RSV-infected mice. BALB/c mice (n=5 per group) were administered either water (vehicle) or Formula (II) (10 mg/kg) intratracheally. 16 hrs after administration, the mice were infected with RSV in the presence of either Formula (II) or water. At 2 days post-RSV infection, lungs were collected and the extent of NOD2 expression in the respiratory tract was examined by RT-PCR. GADPH expression served as a loading control. Each lane for RSV+water and RSV+SB represents the results from a single mouse, and the data is representative of three mice per group.

FIGS. 7A-7F depicts fixed lung sections of RSV-infected mice treated with Formula (II). BALB/c mice (n=5 per group) were administered either water (vehicle) or Formula (II) (10 mg/kg) intratracheally. 16 hrs after administration, the mice were infected with RSV in the presence of either Formula (II) or water. At either 2 days or 4 days post-RSV infection (p.i), lungs were collected, and lung sections were fixed and stained with H&E.

FIG. 8 depicts a graph showing the anti-RSV activity of Formula (II) in the lungs of mice. BALB/c mice (n=5) were infected with RSV intratracheally. At 16 hrs post-RSV infection, the mice were administered either water (vehicle) or Formula (II) (20 mg/kg). A second dosage of either vehicle or Formula (II) was administered 24 hrs after the first dosage. After 24 hrs (64 hrs post-RSV infection), lungs from the infected mice were harvested and the resulting homogenate was subjected to a plaque assay to determine the RSV titer in the lung.

FIG. 9 depicts a plaque assay showing the anti-RSV activity of Formula (II) in the lungs of mice. BALB/c mice (n=5) were infected with RSV intratracheally. At 16 hrs post-RSV infection, the mice were administered either water (vehicle) or Formula (II) (20 mg/kg). A second dosage of either vehicle or Formula (II) was administered 24 hrs after the first dosage. After 24 hrs (64 hrs post-RSV infection), lungs from the infected mice were harvested and the resulting homogenate was subjected to a plaque assay to determine the RSV titer in the lung. Each well represents the results from a single mouse.

FIG. 10 depicts a graph illustrating reduced IFN-β levels in the lungs of RSV-infected mice following treatment with Formula (II). BALB/c mice (n=5) were infected with RSV intratracheally. At 16 hrs post-RSV infection, the mice were administered either water (vehicle) or Formula (II) (20 mg/kg). A second dosage of either vehicle or Formula (II) was administered 24 hrs after the first dosage. After 24 hrs (64 hrs post-RSV infection), lungs from the infected mice were harvested and the resulting homogenate was subjected to an ELISA assay to quantify the level of IFN-β in the lungs.

FIG. 11 depicts a graph illustrating reduced TNF levels in the lungs of RSV-infected mice following treatment with Formula (II). BALB/c mice (n=5) were infected with RSV intratracheally. At 16 hrs post-RSV infection, the mice were administered either water (vehicle) or Formula (II) (20 mg/kg). A second dosage of either vehicle or Formula (II) was administered 24 hrs after the first dosage. After 24 hrs (64 hrs post-RSV infection), lungs from the infected mice were harvested and the resulting homogenate was subjected to an ELISA assay to quantify the level of TNF in the lungs.

FIG. 12. depicts a graph illustrating the anti-RSV activity of compound shown in Formula IV. Viral infection of human lung epithelial (HLE) A549 cells. HLE cells were pre-treated with vehicle or Compound IV (20 μM) for 16 h. The cells were then infected with RSV (0.5 MOI) for 18 h in the presence of either vehicle or compound IV. HLE cells were infected with RSV in serum free antibiotic free OPTI-MEM medium (GIBCO). Following adsorption for 1.5 h at 37° C., cells were washed twice with serum containing DMEM and the infection was continued in the presence of serum (+/−IV). Infectious viral titer was calculated by plaque assay analysis of the medium supernatant from infected cells. Plaque assay was performed by using CV-1 cells.

FIGS. 13A and B. depict graphs illustrating the prophylactic and therapeutic anti-RSV activity of compound shown in Formula VI. Viral infection of human lung epithelial (HLE) A549 cells. For prophylactic activity, HLE cells were pre-treated with vehicle or Compound VI (20 μM) for 16 h. The cells were then infected with RSV (0.5 MOI) for 18 h in the presence of either vehicle or compound VI. HLE cells were infected with RSV in serum free antibiotic free OPTI-MEM medium (GIBCO). Following adsorption for 1.5 h at 37° C., cells were washed twice with serum containing DMEM and the infection was continued in the presence of serum (+/−VI). Infectious viral titer was calculated by plaque assay analysis of the medium supernatant from infected cells. Plaque assay was performed by using CV-1 cells. For demonstration of therapeutic activity, HLE cells were infected with RSV and 24 hours later, cells were treated with compound VI and viral titer determined as described before.

FIG. 14. Depicts the Anti-RSV activity of the isomeric Formula IIb and IIc (tartaric acid salt forms) in vitro in HLE (A549) cells. HLE cells were pre-treated with vehicle or IIc or IIc tartrate salts at (20 μM) for 16 h. The cells were then infected with human RSV for 16 h in the presence of either vehicle or the isomers. Infectious viral titer was calculated by plaque assay analysis of the medium supernatant from infected cells. *p and **p<0.05 were calculated using Student's t-test.

FIG. 15 depicts the anti-flu activity of Formula IIa. A549 cells were pre-treated with vehicle or IIa at 40 μM for 16 h. (In the Fig. SB4367 or SB438 represent two different synthetic lots of IIa). Cells were then infected with flu (influenza virus strain A/Puerto Rico/8/1934 H1N1) (1 MOI or 2 MOI) for 24 h in the presence of either vehicle or Ha. Flu infection was investigated by analyzing expression of flu hemagglutinin (HA) gene expression by RT-PCR. GAPDH expression served as a loading control.

### DETAILED DESCRIPTION OF THE INVENTION

The present invention relates to methods of treating a subjected infected with a viral infection, for example, RSV, influenza, adenovirus, or rhinovirus, the method comprising administration of a compound described herein, e.g. a compound Formula I-VI or pharmaceutically acceptable salt thereof.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy, e.g. LC-MS.

As used herein, an amount of a compound, conjugate, or substance effective to treat a disorder (e.g., a disorder described herein), “therapeutically effective amount,” “effective amount” or “effective course” refers to an amount of the compound, substance, or composition which is effective, upon single or multiple dose administration(s) to a subject, in treating a subject, or in curing, alleviating, relieving or improving a subject with a disorder (e.g., a microbial infection) beyond that expected in the absence of such treatment.

As used herein, the terms “prevent” or “preventing” as used in the context of a disorder or disease, refer to administration of an agent to a subject, e.g., the administration of a compound of the present invention (e.g., compound of Formula (I) or a prodrug (e.g., a compound of Formula (II-VI)) to a subject, such that the onset of at least one symptom of the disorder or disease is delayed as compared to what would be seen in the absence of administration of said agent.

As used herein, the term “subject” is intended to include human and non-human animals. Exemplary human subjects include a human patient having a disorder, e.g., a disorder described herein, or a normal subject. In some embodiments, the subject is a human, e.g., a child or an adult. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc. In some embodiments, the subject is a mouse (e.g., a BALB/c mouse).

As used herein, the terms “treat” or “treating” a subject having a disorder or disease refer to subjecting the subject to a regimen, e.g., the administration of a compound of Formula (I) or a prodrug (e.g., a compound of Formula (II-VI)) or pharmaceutically acceptable salt thereof, or a composition comprising Formula (I) or a prodrug (e.g., a compound of Formula (II-VI)) or pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease.

Numerous ranges, e.g., ranges for the amount of a drug administered per day, are provided herein. In some embodiments, the range includes both endpoints. In other embodiments, the range excludes one or both endpoints. By way of example, the range can exclude the lower endpoint. Thus, in such an embodiment, a range of 250 to 400 mg/day, excluding the lower endpoint, would cover an amount greater than 250 that is less than or equal to 400 mg/day.

### Compounds

The present disclosure features methods for the treatment of a viral infection or a complication of an infection. Exemplary viral infections include RSV, influenza, adenovirus, and rhinovirus infection in a subject, comprising administration of a compound of Formula (I) or a prodrug or pharmaceutically acceptable salt thereof (e.g., a salt described herein such as a tartrate salt). The active agent is Formula (I), which may be described by any one of Formula (Ia), Formula (Ib), and Formula (Ic), or a combination thereof:

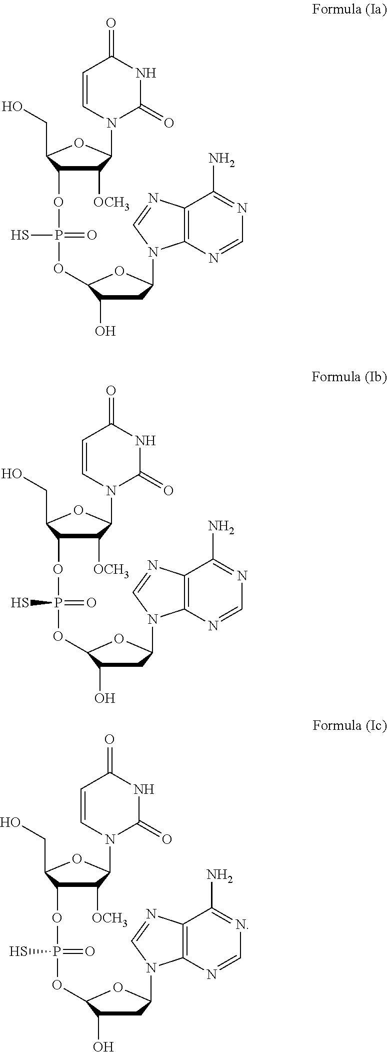

In an embodiment, the prodrug is a compound of Formula (P)

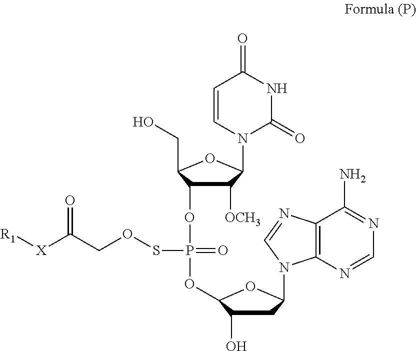

wherein,

X is a bond, O, NH, or S; and

R¹ is optionally substituted alkyl or heteroalkyl.

In an embodiment, X is a bond and R¹ is alkyl (e.g., t-butyl). In an embodiment, X is a bond and R¹ is heteroalkyl (e.g., an optionally substituted heteroalkyl).

In an embodiment, X is O and R¹ is alkyl or heteroalkyl.

The composition of the present invention may comprise a prodrug of Formula (I), wherein said prodrug is a compound of Formula (II). The prodrug (e.g., the compound of Formula (II)) may be described by any one of Formula (IIa), Formula (IIb), and Formula (IIc), or a combination thereof:

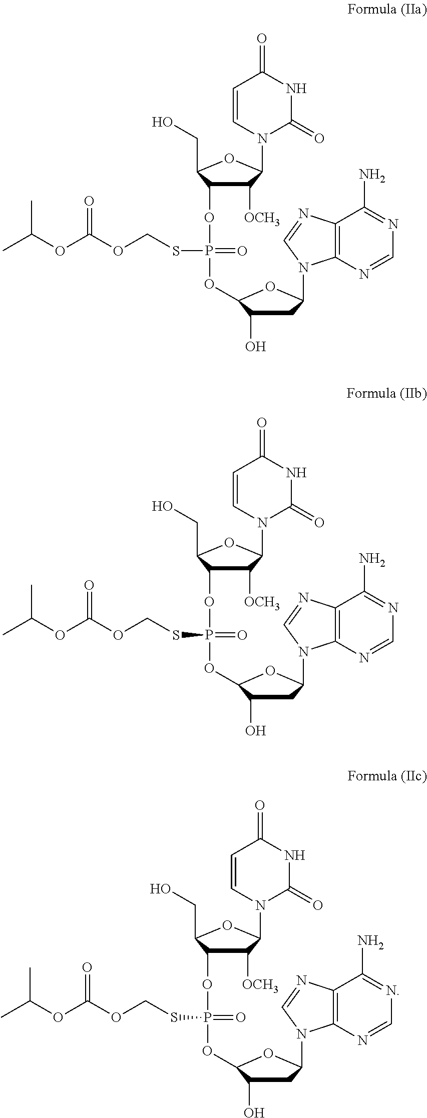

In an embodiment, the prodrug of Formula (I) is a compound of Formula (III), Formula (IV), Formula (V), or Formula (VI):

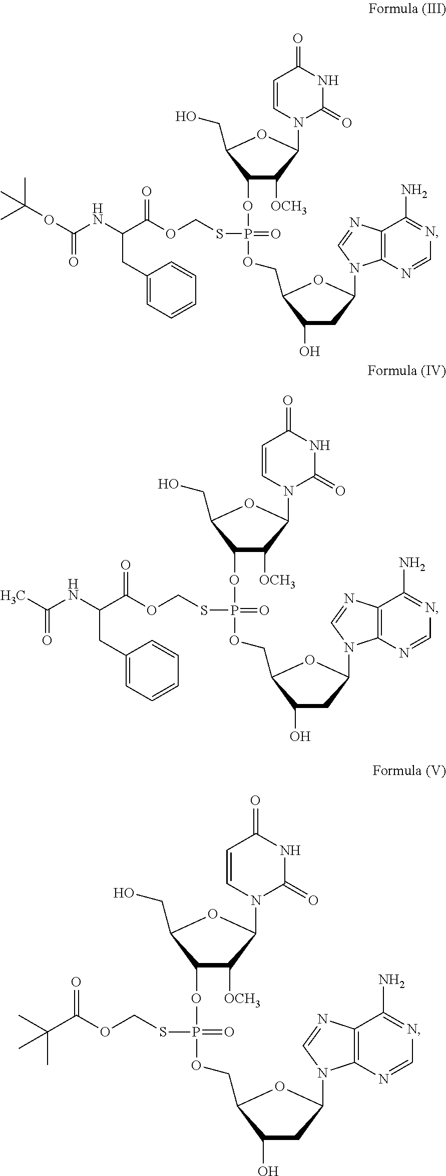

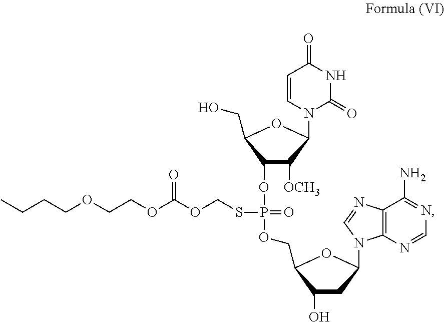

or a pharmaceutically acceptable salt thereof.

In an embodiment the compound is a pharmaceutically acceptable salt such as a salt described herein (e.g., a tartrate salt).

In an embodiment, a compound described herein is administered in an enriched formulation for a single isomer (e.g., an enriched formulation for an Rp or Sp isomer). Example relative ratios and percentages of enrichment are described above. In some embodiments, the individual isomeric forms of the prodrugs represented by Formulas III-VI may be employed, for example, as described in the ratios and percentages described above for compounds of Formula (I) and (II).

Formula (I) and its prodrug Formula (II) are small molecule nucleic acid hybrid (dinucleotide) compounds that combine both antiviral and immune modulating activities. The latter activity mediates controlled apoptosis of virus-infected cells via stimulation of the innate immune response, similar to what is also achieved by IFN-α therapy in patients suffering from a viral infection.

Without wishing to be bound by theory, the mechanism of action of Formula (I) and its prodrugs described herein (e.g., Formulae (II-VI) may be dissected into two components. The first component entails the host immune stimulating activity of Formula (I), which induces endogenous IFNs via the activation of viral sensor proteins, e.g., retinoic acid-inducible gene 1 (RIG-I) and nucleotide-binding oligomerization domain-containing protein 2 (NOD2) (Takeuchi, O. and Akira S. Cell (2010) 140:805-820; Sato, S. et al. Immunity (2015) 42:123-132; Sabbah, A. et al. Nat Immunol (2009) 10:1073-1080). Activation may occur by binding of Formula (I) to the RIG-I/NOD2 proteins at their nucleotide binding domain. The RIG-I and NOD2 proteins are located in the cytosol of cells, including airway epithelial cells, and usually recognize signature patterns of foreign nucleic acids such as the pathogen associated molecular pattern (PAMP). Once PAMP within viral RNA or DNA is recognized, RIG-I and NOD2 may become activated and trigger the IFN signaling cascade that then results in IFN and interferon-stimulated gene (ISG) production and induction of an antiviral state in cells. In the case of RSV, the PAMP is believed to be the single-stranded genomic RNA.

The second component of the mechanism of action of Formula (I) and a prodrug such as a compound of Formula (II-VI) involves its direct antiviral activity, which inhibits the synthesis of viral nucleic acids by steric blockage of the viral polymerase. The block may be achieved by interaction of Formula (I) with RIG-I and NOD2 as described above that then in turn may prevent the polymerase enzyme from engaging with the viral nucleic acid template for replication (i.e, RSV RNA). The cytotoxic potential of a prodrug such as a compound of Formula (II-VI) has been initially evaluated using a panel of cell lines. Similar to the parental drug, Formula (II) demonstrated an excellent safety profile, with a 50% cytotoxic concentration (CC50) of greater than 1000 μM (Coughlin, J. E. et al. Bioorg Med Chem Lett (2010) 20:1783-1786). Formula (II) has been further evaluated for anti-HBV activity in a cell-based assay against wild-type HBV and against lamivudine-(3TC) and adefovir-(ADV) resistant mutant HBV, and has also exhibited potency against HCV and other viral infections.

In some embodiments, the method described herein comprises administration of a compound of Formula (I) or a pharmaceutically acceptable salt thereof. In other embodiments, the method described herein comprises administration of prodrug of Formula (I) (e.g., a compound of Formula (II-VI)) or a pharmaceutically acceptable salt thereof. In other embodiments, the method herein describes administration of a composition comprised of a combination of a compound of Formula (I) and a prodrug thereof, such as a compound of Formula (II-VI) or pharmaceutically acceptable salts thereof (e.g., a tartrate salt). It is well established that the prodrug Formula (II) has been shown to be converted to the active drug Formula (I) (e.g., the Rp- and Sp isomers, e.g., Formula (Ib) and Formula (Ic)) upon administration.

The compounds provided herein may contain one or more asymmetric centers and thus occur as racemates and racemic mixtures, single enantiomers, individual diastereomers and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

### Viral Infections

The present invention relates to the treatment of a viral infection. Exemplary viral infections include Respiratory Syncytial Virus (RSV), Influenza, Parainfluenza virus and Rhinovirus. The present disclosure relates to methods for treating a subject infected with RSV through administration of Formula (I)-(VI) or the prodrug Formula (II), or a pharmaceutically acceptable salt thereof (e.g., a tartrate salt).

RSV is an enveloped RNA virus that is a member of the Paramyxoviridae family of viruses. The viral genome is composed of single-stranded RNA with negative polarity, and contains 10 genes that encode for 11 proteins. The virus is characterized into two major serotypes (serotypes A and B) based upon the antigenic epitopes present on the viral envelope proteins F (fusion) and G (glycoprotein). In some embodiments, the methods described herein are used to treat a subject suffering from any known form of RSV infection (e.g., any genotype or serotype of RSV or a combination or variant thereof).

RSV is highly contagious and typically infects a subject through infected nasal and oral fluids. Once present in a subject, the virus rapidly spreads along the epithelium of the respiratory tract through cell to cell transfer. Upon reaching the lower respiratory tract, RSV often induces cause bronchiolitis or pneumonia in the subject, which may coexist with RSV infection. The typical incubation period of RSV is between 2-8 days, more often between 3-6 days. RSV frequently infects small children, and it is estimated that nearly all children have been infected with the virus by the age of three. In adults, RSV infection poses a similar disease burden to influenza in high-risk individuals including the elderly, and accounts for roughly 10,000 deaths per year in the United States among over 65 years of age.

There are currently only two approved treatments for RSV infection. The first, the monoclonal antibody palivizumab, is provided as a prophylactic therapy for young children at risk, such as premature infants and those suffering from chronic heart and lung infections. The second treatment is the antiviral agent ribavirin, which is believed to interfere with the expression of viral mRNA. However, treatment with ribavirin is not universally successful and is associated with toxicity at high doses or with long term use. Therefore, there is an urgent need for a new class of RSV therapies that can neutralize the virus in patients with weakened immune systems without inducing toxicity and viral resistance.

Despite the availability of vaccine, influenza A virus (flu) epidemics occur annually with 3-5 million severe cases in the young, elderly, and those with serious medical conditions, resulting in up to 500,000 deaths/year. The two main classes of antiviral drugs used against influenza are neuraminidase inhibitors, such as zanamivir and oseltamivir, or inhibitors of the viral M2 protein, such as amantadine and rimantadine. These drugs can reduce the severity of symptoms if taken soon after infection and can also be taken to decrease the risk of infection. However, virus strains have emerged that show drug resistance to both classes of drug. Furthermore, antiviral drugs such as oseltamivir, and zanamivir are only marginally effective and are ineffective in patients with flu complication.

There are three types of influenza viruses called Type A, Type B, and Type C. Influenza viruses are (−) strand RNA viruses because of the polarity of the RNA that is carried in the virion.

Human parainfluenza 3 (HPIV3) is one of the major respiratory RNA viruses that causes severe respiratory airway disease in infants and children and there is no vaccine for HPIV3. Human parainfluenza viruses (hPIVs) are the viruses that cause ‘human parainfluenza.’ hPIVs are a group of four distinct serotypes of enveloped single-stranded RNA viruses belonging to the paramyxovirus family.

Rhinoviruses cause the common colds. There are 99 recognized types of human rhinoviruses that differ according to their surface proteins (serotypes). They are lytic in nature and are among the smallest viruses with diameters of about 30 nanometers. Rhinoviruses have single-stranded positive sense RNA genomes of between 7200 and 8500 nt in length. At the 5′ end of the genome is a virus-encoded protein, and carry a 3′ poly-A tail. Structural proteins are encoded in the 5′ region of the genome and non-structural at the 3′ end. The viral particles are not enveloped and are icosahedral in structure.

### Pharmaceutical Compositions

The present invention features methods for treating a subject infected with RSV, the methods comprising administering a compound of Formula (I) or a prodrug thereof (e.g., a compound of Formula (II-VI)), or a pharmaceutically acceptable salt thereof.

While it is possible for the compound of the present invention (e.g., a compound of Formula (I) or a prodrug thereof (e.g., a compound of Formula (II-VI)) to be administered alone, it is preferable to administer said compound as a pharmaceutical composition or formulation, where the compounds are combined with one or more pharmaceutically acceptable diluents, excipients or carriers. The compounds according to the invention may be formulated for administration in any convenient way for use in human or veterinary medicine. In certain embodiments, the compounds included in the pharmaceutical preparation may be active itself, or may be a prodrug, e.g., capable of being converted to an active compound in a physiological setting (e.g., a compound of Formula (II-VI)). Regardless of the route of administration selected, the compounds of the present invention, which may be used in a suitable hydrated form, and/or the pharmaceutical compositions of the present invention, are formulated into a pharmaceutically acceptable dosage form such as described below or by other conventional methods known to those of skill in the art.

The amount and concentration of compounds of the present invention (e.g., a compound of Formula (I) or a prodrug thereof (e.g., a compound of Formula (II-VI)) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present invention provides pharmaceutically acceptable compositions comprising a therapeutically effective amount or prophylacticaly effective amount of a compound described herein (e.g., a compound of Formula (I) or a prodrug thereof (e.g., a compound of Formula (II-VI)), formulated together with one or more pharmaceutically acceptable carriers (additives) and/or diluents. As described in detail below, the pharmaceutical compositions of the present invention may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water; (18) isotonic saline; (19) Ringer's solution; (20) ethyl alcohol; (21) phosphate buffer solutions; (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming pharmaceutically acceptable salts with pharmaceutically acceptable acids. The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein. For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present invention may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, powder, dragée, or powder. The pharmaceutical composition may be in unit dosage forms suitable for single administration of precise dosages. Pharmaceutical compositions may comprise, in addition to the compound described herein (e.g., a compound of Formula (I) or a prodrug thereof (e.g., a compound of Formula (II-VI)) or a pharmaceutically acceptable salt thereof, a pharmaceutically acceptable carrier, and may optionally further comprise one or more pharmaceutically acceptable excipients, such as, for example, stabilizers (e.g., a binder, e.g., polymer, e.g., a precipitation inhibitor, diluents, binders, and lubricants.

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising a compound of the present invention (e.g., a compound of Formula (I) or a prodrug thereof (e.g., a compound of Formula (II-VI)) or a pharmaceutically acceptable salt thereof.

Formulations of the present invention include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this invention suitable for parenteral administration comprise compounds of the invention in combination with one or more pharmaceutically acceptable sterile isotonic aqueous or nonaqueous solutions, dispersions, suspensions or emulsions, or sterile powders which may be reconstituted into sterile injectable or inhalable solutions or dispersions just prior to use, which may contain antioxidants, buffers, bacteriostats, solutes which render the formulation isotonic with the blood of the intended recipient or suspending or thickening agents.

In some embodiments, the composition described herein comprises a liquid dosage form for administration by inhalation, e.g., a solution, a suspension, an emulsion, a spray, a mist, or an aerosol. In some embodiments, the composition described herein comprises a liquid dosage form for intratracheal administration, e.g., a solution, a suspension, an emulsion, a spray, a mist, or an aerosol. In some embodiments, the spray, mist, or aerosol comprises particles between about 0.01 microns to about 10 microns in size. In some embodiments, the spray, mist, or aerosol comprises particles of about 0.01 micron, about 0.025 micron, about 0.05 micron, about 0.075 micron, about 0.1 micron, about 0.25 micron, about 0.5 micron, about 0.75 micron, about 1 micron, about 1.25 microns, about 1.5 microns, about 1.75 microns, about 2 microns, about 3 microns, about 4 microns, about 5 microns, about 6 microns, about 7 microns, about 8 microns, about 9 microns, or about 10 microns in size, although the actual size of the particles may be outside of this range.

In some embodiments, the composition described herein comprises a solid dosage form for administration by inhalation, e.g., granules, particles, a powder, a gel, or a microencapsulated dosage form. In some embodiments, the composition described herein comprises a solid dosage form for intratracheal administration, e.g., granules, particles, a powder, a gel, or a microencapsulated dosage form. In some embodiments, the granules, particles, powder, gel, or microencapsulated dosage form comprises particles between about 0.01 microns to about 10 microns in size. In some embodiments, the granules, particles, powder, gel, or microencapsulated dosage form comprises particles of about 0.01 micron, about 0.025 micron, about 0.05 micron, about 0.075 micron, about 0.1 micron, about 0.25 micron, about 0.5 micron, about 0.75 micron, about 1 micron, about 1.25 microns, about 1.5 microns, about 1.75 microns, about 2 microns, about 3 microns, about 4 microns, about 5 microns, about 6 microns, about 7 microns, about 8 microns, about 9 microns, or about 10 microns in size, although the actual size of the particles may be outside of this range.

In addition, said dosage forms and formulations may include other medicinal or pharmaceutical agents, carriers, excipients, and/or adjuvants. Exemplary pharmaceutical compositions include compressed tablets (e.g., directly compressed tablets), e.g., comprising a compound of the present invention (e.g., a compound of Formula (I) or a prodrug thereof (e.g., a compound of Formula (II-VI)) or a pharmaceutically acceptable salt thereof.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable or inhalable pharmaceutical form may be brought about by the inclusion of agents that delay absorption such as aluminum monostearate and gelatin.

In some cases, in order to prolong the effect of a compound of the present invention (e.g., a compound of Formula (I) or a prodrug thereof (e.g., a compound of Formula (II-VI), it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered form of the compound of the present invention is accomplished by dissolving or suspending compound in an oil vehicle.

In some embodiments, it may be advantageous to administer the compound of the present invention (e.g., a compound of Formula (I) or a prodrug thereof (e.g., a compound of Formula (II-VI)) in a sustained fashion. It will be appreciated that any formulation that provides a sustained absorption profile may be used. In certain embodiments, sustained absorption may be achieved by combining a compound of the present invention with other pharmaceutically acceptable ingredients, diluents, or carriers that slow its release properties into systemic circulation.

### Routes of Administration

The compounds and compositions used in the methods described herein may be administered to a subject in a variety of forms depending on the selected route of administration, as will be understood by those skilled in the art. Exemplary routes of administration of the compositions used in the methods described herein include inhalation, intratracheal, intranasal, topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, intratracheal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time. In certain embodiments of the invention, the compositions described herein comprising a compound of Formula (I) or a prodrug thereof (e.g., a compound of Formula (II-VI)) is administered orally. In other embodiments of the invention, the compositions described herein comprising a compound of Formula (I) or a prodrug thereof (e.g., a compound of Formula (II-VI)) is administered parenterally (e.g., intraperitoneally).

For intravenous, intraperitoneal, intrathecal, or intratracheal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

In some embodiments, the methods of the present invention feature administration of a compound of Formula (I) or Formula (II-VI) through inhalation or by intratracheal delivery. In some embodiments, the compound or a composition thereof may be administered through a nebulizer or inhaler (e.g., a dry powder inhaler or a metered dose inhaler).

### Dosages

The compositions of the present invention are formulated into acceptable dosage forms by conventional methods known to those of skill in the art. Actual dosage levels of the active ingredients in the compositions of the present invention (e.g., a compound of Formula (I) or a prodrug thereof (e.g., a compound of Formula (II-VI)) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or intraperitoneally) to a subject afflicted with the disorders described herein (e.g., RSV infection). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or intraperitoneally) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Patient Selection and Monitoring

The methods of the present invention described herein entail administration of a compound of Formula (I) or a prodrug thereof (e.g., a compound of Formula (II-VI)) or a pharmaceutically acceptable salt thereof for the treatment of RSV infection. Accordingly, a patient and/or subject can be selected for treatment using a compound of Formula (I) or a prodrug thereof (e.g., a compound of Formula (II-VI)) or a pharmaceutically acceptable salt thereof by first evaluating the patient and/or subject to determine whether the subject is infected with RSV and determination of the serotypic and genotypic classification of the virus. A subject can be evaluated as infected with RSV using methods known in the art. The subject can also be monitored, for example, subsequent to administration of a compound described herein (e.g., a compound of Formula (I) or a prodrug thereof (e.g., a compound of Formula (II-VI)) or a pharmaceutically acceptable salt thereof.

In some embodiments, the subject is a mammal. In some embodiments, the subject is a human. In some embodiments, the subject is an adult (e.g., over about 18 years of age, over about 35 years of age, over about 65 years of age, over about 80 years of age). In some embodiments, the subject is a child (e.g., under about 5 years of age, under about 4 years or age, under about 3 years of age, under about 2 years of age, under about 1 year of age). In some embodiments, the subject is suffering from a severe RSV infection. In some embodiments, the subject is immunocompromised (e.g., a subject that may have a weakened immune system relative to a reference standard, or may be suffering from an immune disease or condition). In some embodiments, the subject is suffering from bronchiolitis, pneumonia, or other respiratory illness or condition. In some embodiments, the subject is suffering from a cardiac illness or condition. In some embodiments, the subject is suffering with a bacterial infection.

In some embodiments, the subject is treatment naïve. In some embodiments, the subject has been previously treated for RSV infection (e.g., with an antibody or antiviral agent). In some embodiments, the subject has previously been infected with or diagnosed with RSV infection. In some embodiments, the subject has been previously treated for an immunodeficiency (e.g., with an antibacterial agent, an antiviral agent, antibody, protein therapeutic, or other agent). In some embodiments, the subject has been previously treated or is currently being treated for bronchiolitis, pneumonia, or other respiratory illness or condition. In some embodiments, the subject has been previously treated or is currently being treated for a cardiac illness or condition. In some embodiments, the subject has been previously treated or is currently being treated for a bacterial infection.

In some embodiments, the genotype, serotype, subtype, or antigenic grouping of the RSV infection is known (e.g., RSV-A or RSV-B).

In some embodiments, the subject is a non-human mammal. In some embodiments, the subject is a non-human primate or a rodent. In some embodiments, the subject is a mouse (e.g., a BALB/C mouse), a rat (e.g., a cotton rat), a guinea pig, a pig, a horse, a sheep, a monkey, a baboon, a chimpanzee, a ferret, a woodchuck, or a chinchilla.

### Combination Therapies

In some embodiments, additional therapeutic agents may be administered with compositions of the present invention for the treatment of RSV or any symptom or associated condition thereof. When combination therapy is employed, the additional therapeutic agent(s) can be administered as a separate formulation or may be combined with any of the compositions described herein.

For example, any of the methods described herein may further comprise the administration of a therapeutically effective amount of an additional agent. In some embodiments, the additional agent is an antiviral agent, antibacterial agent, an anticancer agent, anti-inflammatory agent, an antibody, a bronchodilator, an analgesic agent, an antipyretic agent, a cough suppressant, or an antihistamine. In some embodiments, the additional agent is intended to treat a symptom or side effect of RSV infection. In some embodiments, the additional agent is intended to treat a side effect or symptom of the methods presented herein. In some embodiments, more than one additional agent may be administered in combination with the compound of Formula (I) or Formula (II-VI) as described herein.

In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the antiviral agent comprises a capsid inhibitor, an entry inhibitor, a secretion inhibitor, a microRNA, an antisense RNA agent, an RNAi agent, or other agent designed to inhibit viral RNA. In some embodiments, the nucleoside analog comprises entecavir, lamuvidine, adefovir, darunavir, sofosbuvir, telaprevir, tenofovir, zidovudine, ribavirin, lamivudine, entecavir, or AL-8176. In some embodiments, the antiviral agent is ribavirin.

In some embodiments, the anti-inflammatory agent, analgesic agent, or antipyretic agent comprises acetaminophen, aspirin, ibuprofen, naproxen, fenoprofen, dexibuprofen, or ketoprofen. In some embodiments, the cough suppressant (e.g., antitussive agent) comprises carbetapentane, benzonatate, or dextromethorphan. In some embodiments, the antihistamine comprises fexofenadine, loratadine, phenindamine, dexchlorpheniramine, terfenadine, cetirizine, tripolidine, promethazine, diphenhydramine, cyproheptadine, promethazine, carbinoxamine, hydroxyzine, or clemastine. In some embodiments, the anticancer agent comprises methotrexate, 5-fluorouracil, doxorubicin, vincristine, bleomycin, vinblastine, dacarbazine, toposide, cisplatin, epirubicin, and sorafenib tosylate. In some embodiments, the bronchodilator comprises albuterol, salbutamol, epinephrine (e.g., racemic epinephrine), levosalbutamol, pirbuterol, ephedrine, terbutaline, salmeterol, clenbuterol, formoterol, bambuterol, or indacaterol.

### EXAMPLES

### Example 1. Prophylactic Antiviral Activity of Formula (II) Against RSV Infection in Mice

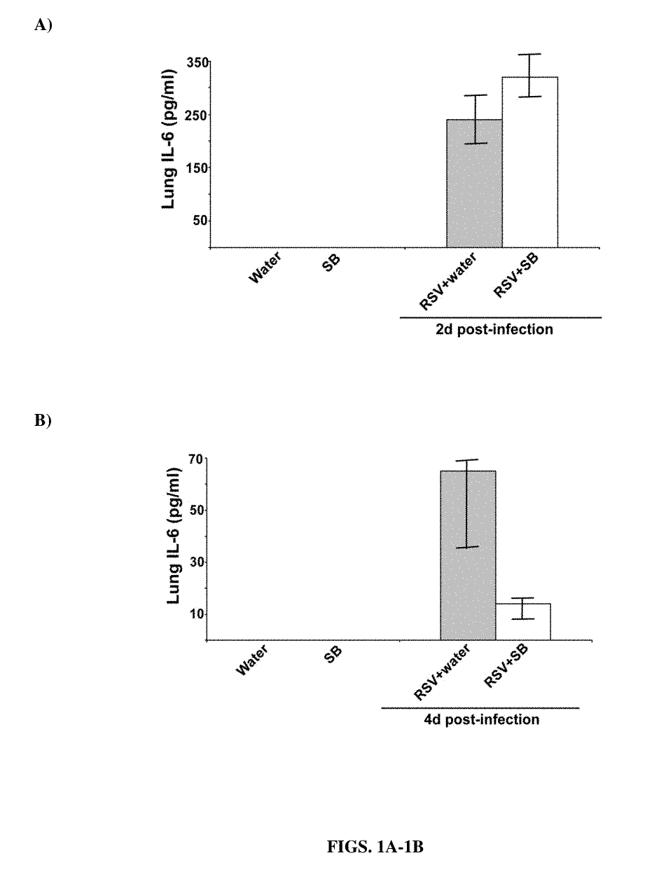

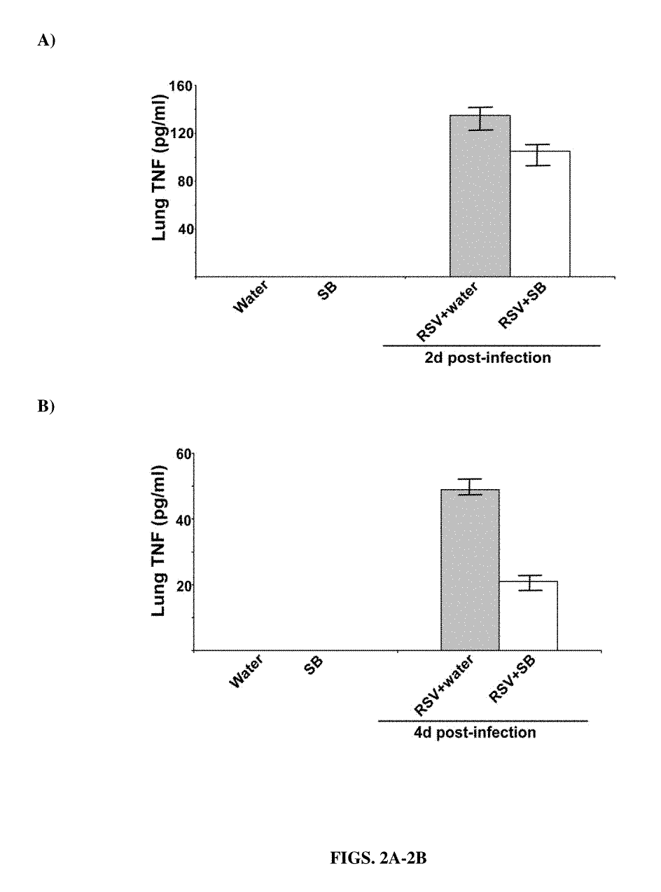

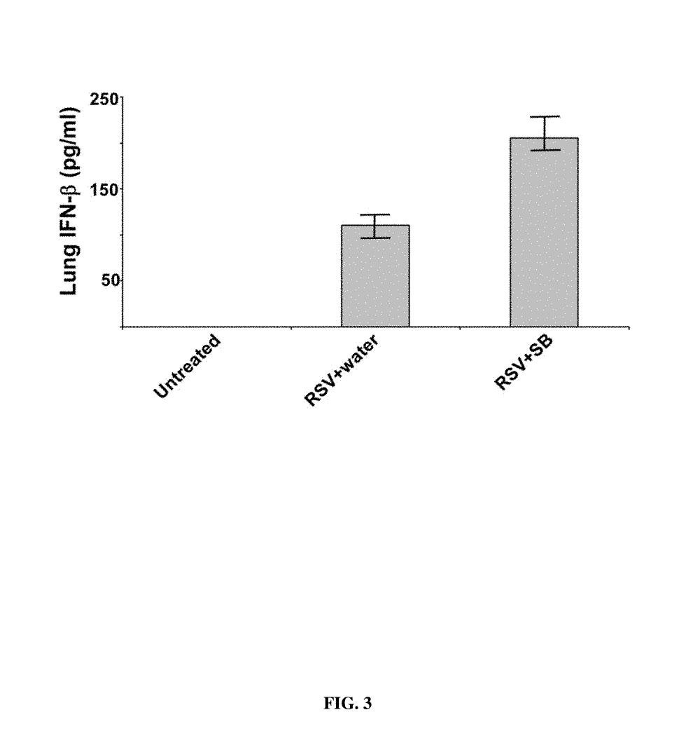

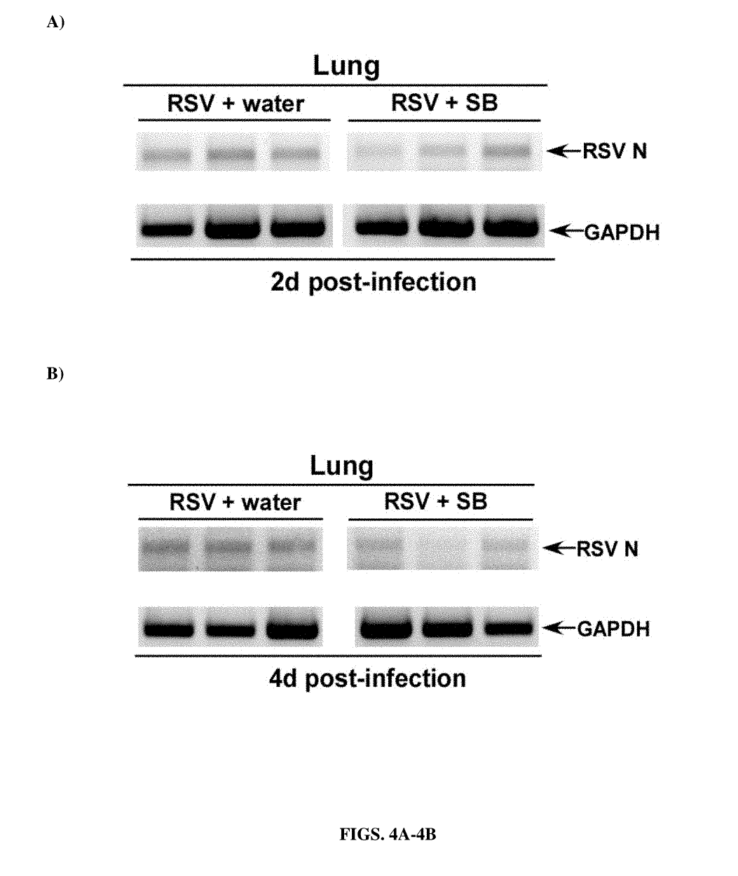

In order to investigate the prophylactic activity of Formula (II) against RSV infection, the levels of multiple downstream signaling markers were determined in mice pre-treated with Formula (II) upon RSV infection. Groups of BALB/c mice (n=5 per group) were administered either water (vehicle) or Formula (II) (10 mg/kg) intratracheally. 16 hours after administration, the mice were infected with RSV in the presence of either Formula (II) or water. At two days or four days post-RSV infection (or both), the lungs were harvested from the mice and levels of IL-6, TNF and IFN-β were determined through ELISA analysis of the lung homogenate. As shown in FIGS. 1A-1B, IL-6 levels increased slightly in Formula (II)-treated mice at two days post-RSV infection, but diminished considerably at 4 days post-infection. TNF levels in the mice were decreased over the four day period (FIGS. 2A-2B), while enhanced IFN-β production was observed in the subjects over the same period (FIG. 3). As all three downstream signaling markers play a significant role in lung inflammation and disease response, these results clearly demonstrate the therapeutic effect of Formula (II) against RSV infection. s

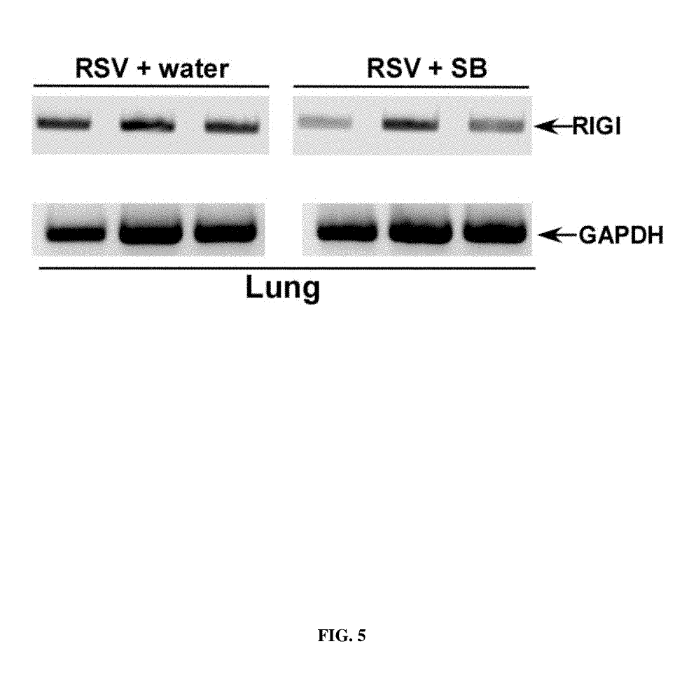

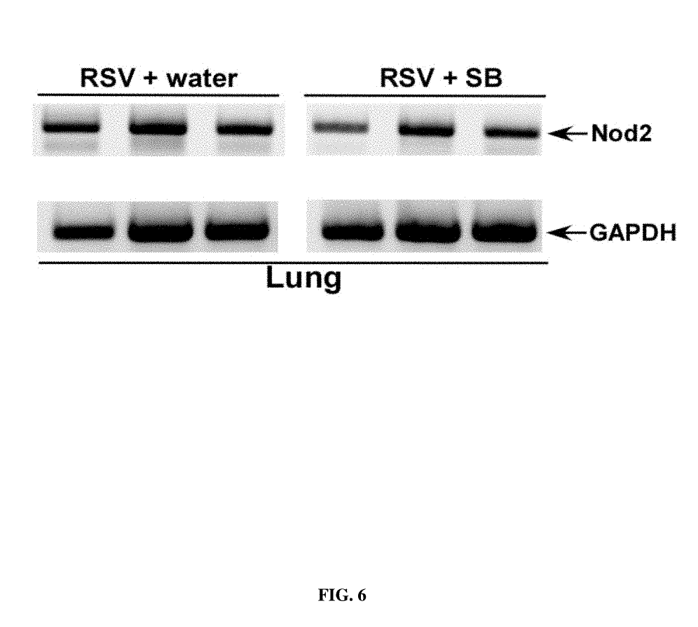

The extent of RSV infection and the levels of RIG-I and NOD2 in the lung homogenate were quantified by RT-PCR. FIGS. 4A-4B shows agarose gel analysis of RSV titer in the mice at both 2 days and 4 days post-infection, confirming the reduced RSV levels present in the airways of Formula (II)-treated mice compared with control subjects. Reduced levels of RIG-I (FIG. 5) and NOD2 (FIG. 6) were observed in these samples as well, which may be explained by the “switching-off” of proinflammatory mediators such as RIG-I and NOD2 in scenarios with lower viral burden.

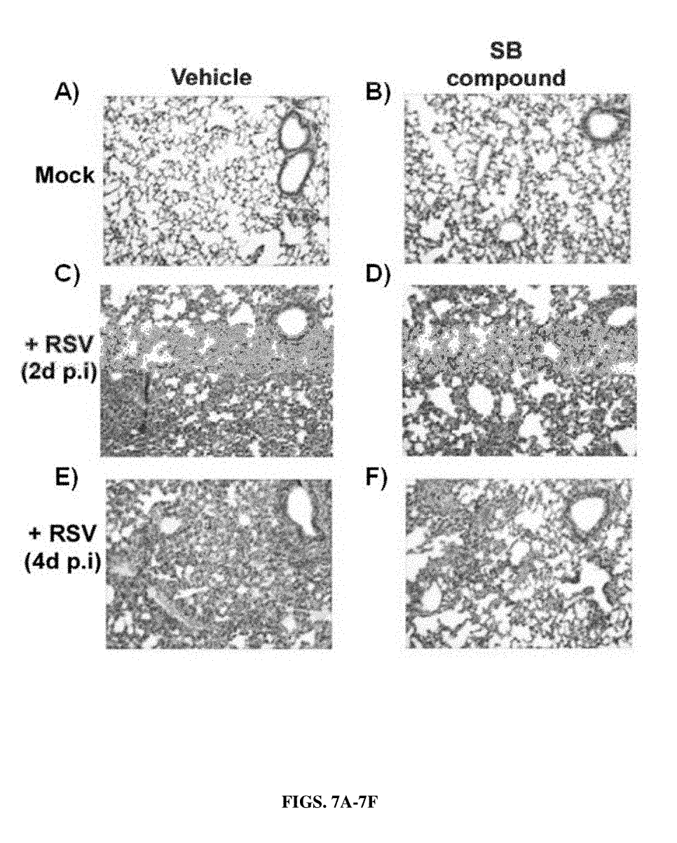

Analysis of the alveolar structure and integrity was carried out through staining of fixed lung sections with H&E (FIGS. 7A-7F). As shown, little to no inflammation was noted in the lungs of Formula (II)-treated uninfected mice. In the untreated RSV-infected mice, high-grade inflammation is observed consistent with known immunopathology of RSV infection. The airways of the RSV-infected mice are filled with infiltrating immune cells and display severe inflammation. In contrast, the RSV-infected mice pre-treated with Formula (II) exhibit reduced inflammation in the lungs, along with many unobstructed airway spaces not observed in the lungs of the untreated subjects. These results indicate that Formula (II) is capable of diminishing disease severity associated with RSV infection.

### Example 2. Prophylactic and Therapeutic Activity of Multiple Formula (II) Doses in RSV-Infected Mice

Groups of BALB/c mice (n=5 per group) were administered either water (vehicle) or Formula (II) (10 mg/kg) intratracheally. As outlined in Example 1, the mice were infected with RSV in the presence of either Formula (II) or water 16 hrs after administration. A second dosage of either vehicle or Formula (II) was administered 24 hrs after the first dosage. After 24 hrs (64 hrs post-RSV infection), lungs from the infected mice were harvested and the resulting homogenate was analyzed to determined the RSV titer and level of multiple downstream signaling markers in the lung. The RSV titer levels were determined by plaque assay, and indicate that the RSV burden is reduced in the lungs of Formula (II) mice. The levels of IFN-β and TNF were determined by ELISA assay and were all diminished in Formula (II)-treated mice compared with the untreated controls, suggesting that Formula (II) does not confer aberrant IFN-β and TNF levels that may contribute to inflammation and tissue damage.

### Example 3. Therapeutic Activity of Multiple Formula (II) Doses in RSV-Infected Mice

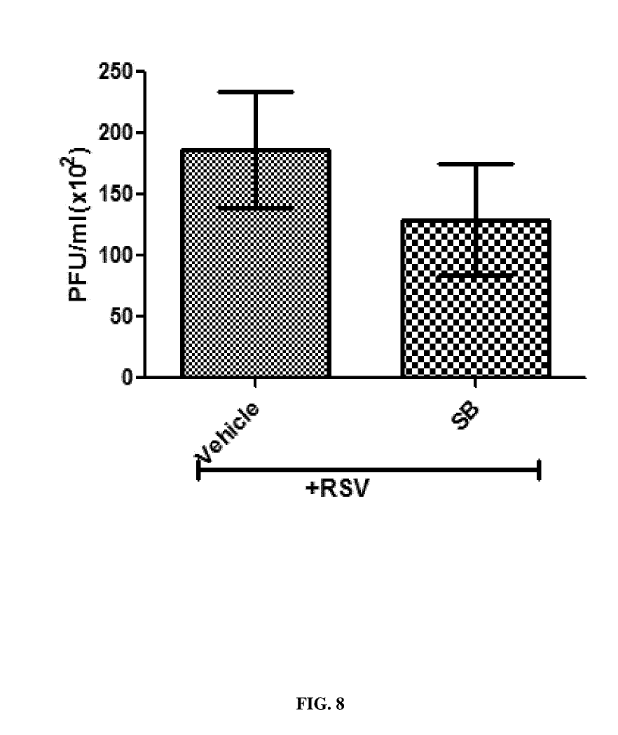

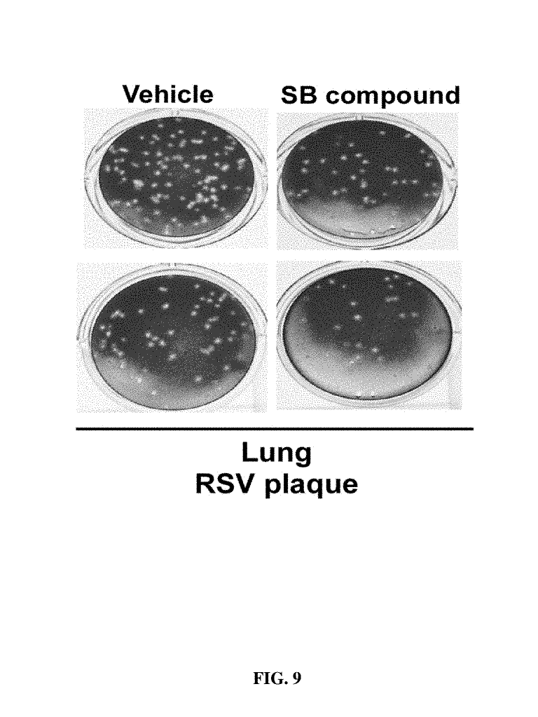

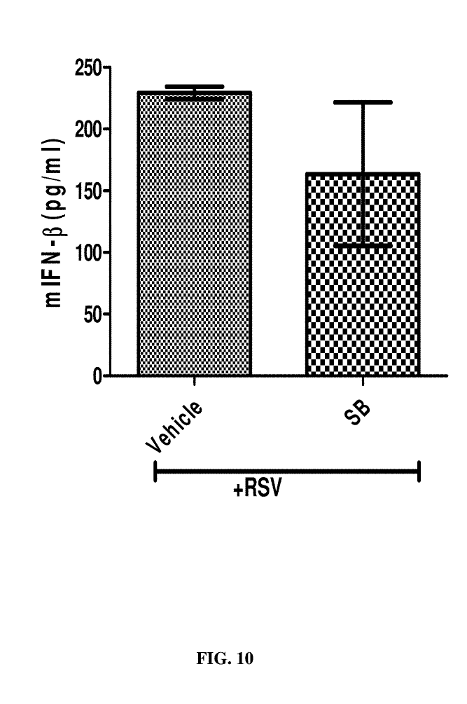

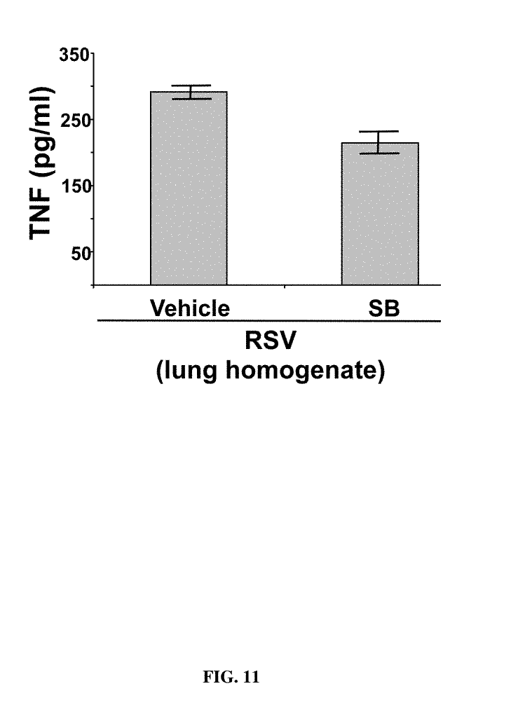

Groups of BALB/c mice (n=5 per group) were administered either water (vehicle) or Formula (II) (10 mg/kg) intratracheally. As outlined in Example 1, the mice were infected with RSV in the presence of either Formula (II) or water 16 hrs after administration. A second dosage of either vehicle or Formula (II) was administered 24 hrs after the first dosage. After 24 hrs (64 hrs post-RSV infection), lungs from the infected mice were harvested and the resulting homogenate was analyzed to determined the RSV titer and level of multiple downstream signaling markers in the lung. The RSV titer levels were determined by plaque assay (FIG. 8 and FIG. 9), and indicate that the RSV burden is reduced in the lungs of Formula (II) mice. The levels of IFN-β (FIG. 10) and TNF (FIG. 11) were determined by ELISA assay and were all diminished in Formula (II)-treated mice compared with the untreated controls, suggesting that Formula (II) does not confer aberrant IFN-β and TNF levels that may contribute to inflammation and tissue damage.

### Example 4. Prophylactic Antiviral Activity of Formula (IV) and Formula (VI) Against RSV Infection

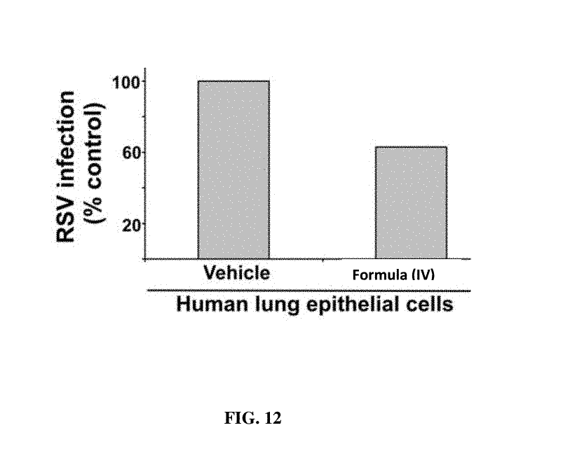

HLE cells were pre-treated with vehicle or Formula (IV) (20 μM) for 16 h. The cells were then infected with RSV for 18 h in the presence of either vehicle or Formula (IV). Infectious viral titer was calculated by plaque assay analysis of the medium supernatant from infected cells. 100% control represents infectious viral titer from vehicle treated (control) cells. We observed anti-RSV activity with Formula (IV) compound (FIG. 12).

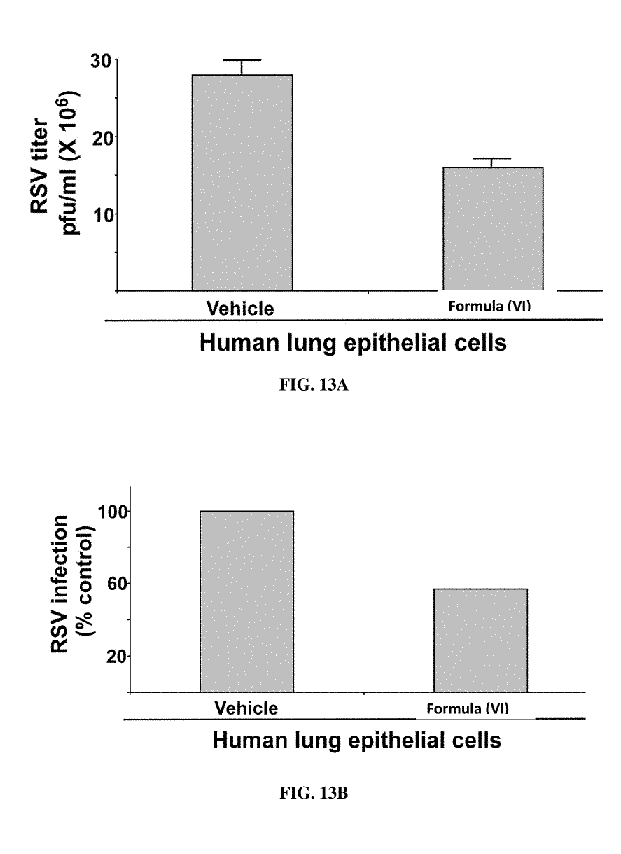

The same experiment was carried out with Formula (VI) (FIGS. 13A-13B), HLE Cells were treated with Formula (VI) (20 micro molar) for 16 h prior to RSV (0.5 MOI) infection and 12 h in the presence of Formula (VI). (FIGS. 13A and 13B).

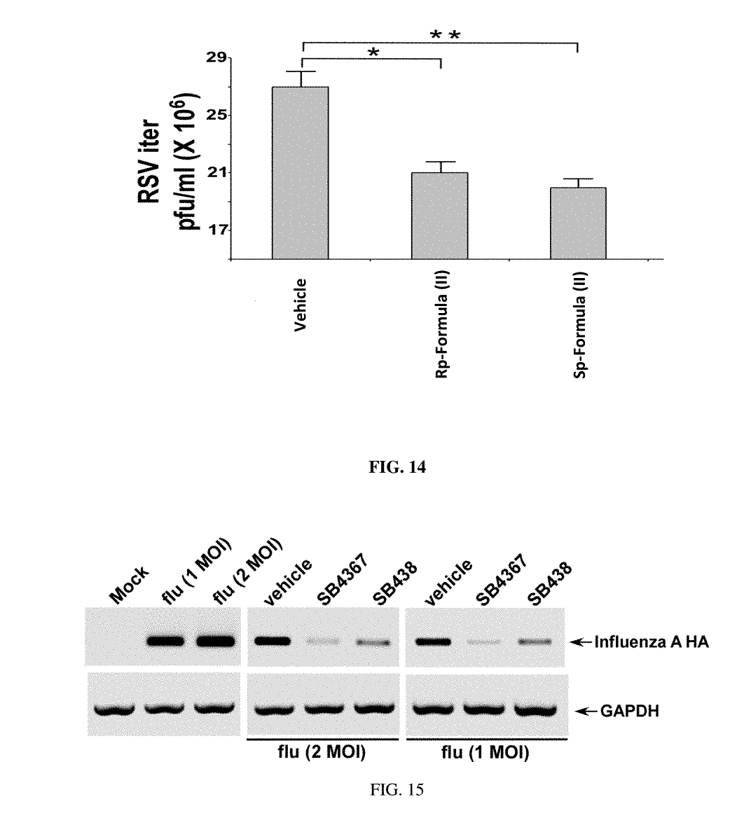

The same experiment was carried out with Rp-Formula (II) and Sp-Formula (II) tartrate salts at (20 μM) for 16 h. Infectious viral titer was calculated by plaque assay analysis of the medium supernatant from infected cells. *p and **p<0.05 were calculated using Student's t-test. The results are depicted in FIG. 14.

### Example 5: Anti-Flu Activity of Formula (II)

A549 cells were pre-treated with vehicle or SB 9200 at 40 μM for 16 h. (In FIG. 15 SB4367 or SB438 represent two different lots of Formula (II)). Cells were then infected with flu (influenza virus strain A/Puerto Rico/8/1934 H1N1) (1 MOI or 2 MOI) for 24 h in the presence of either vehicle or SB 9200. Flu infection was investigated by analyzing expression of flu hemagglutinin (HA) gene expression by RT-PCR. GAPDH expression served as a loading control. The results are depicted in FIG. 15

### EQUIVALENTS

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

What is claimed is:

1. A method for treating a subject infected with respiratory syncytial virus (RSV), influenza, adenovirus or rhinovirus, the method comprising administering to the subject a compound of Formula (I), wherein the compound is selected from: 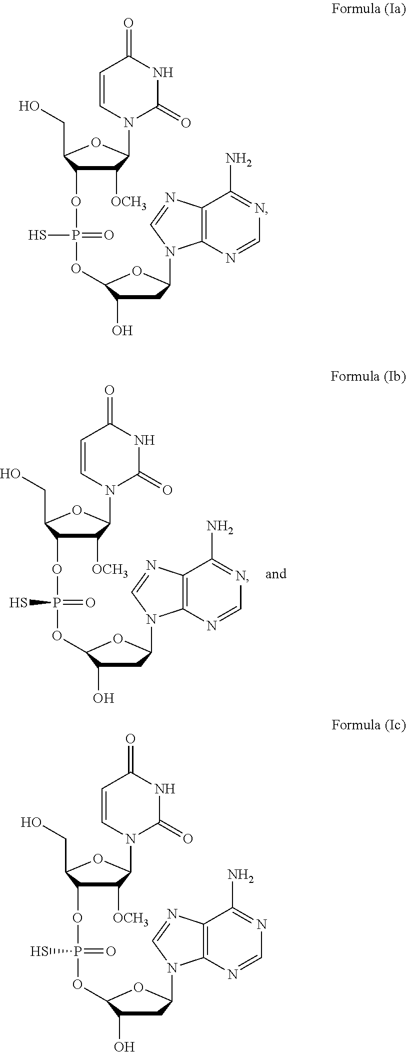
or a prodrug or pharmaceutically acceptable salt thereof.

2-3. (canceled)

4. The method of claim 1, wherein the prodrug of Formula (I) is a compound of Formula (P) 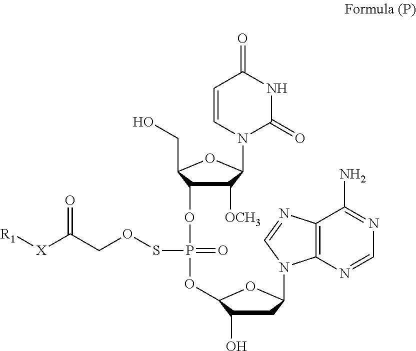
or a pharmaceutically acceptable salt thereof,
wherein,
X is a bond, O, NH, or S; and
R¹ is optionally substituted alkyl or heteroalkyl.

5. The method of claim 1, wherein the prodrug of Formula (I) is a compound of Formula (II), wherein the compound is selected from: 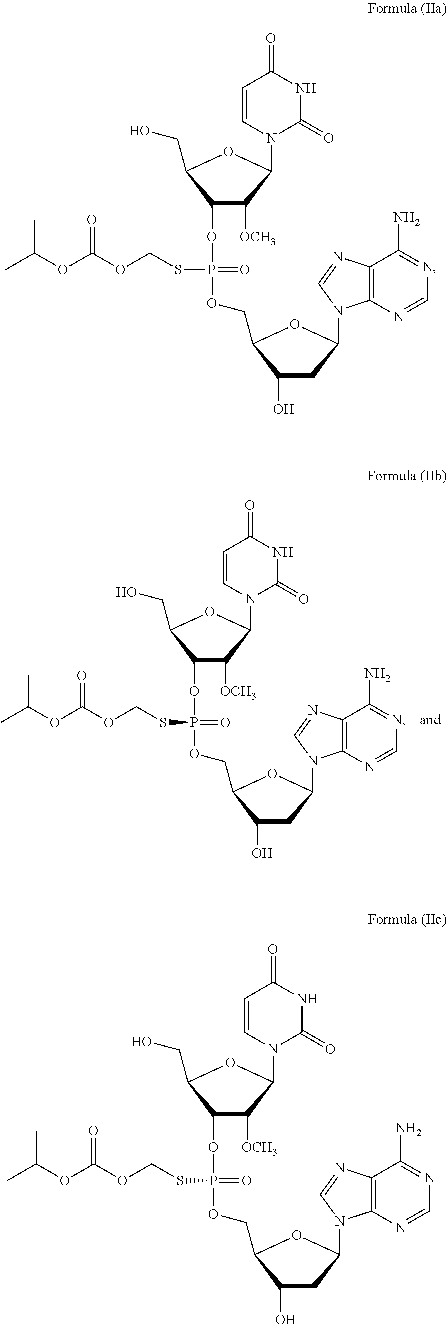
or a pharmaceutically acceptable salt thereof.

6. (canceled)

7. The method of claim 1, wherein the prodrug of Formula (I) is a compound of Formula (III), Formula (IV), Formula (V), or Formula (VI): 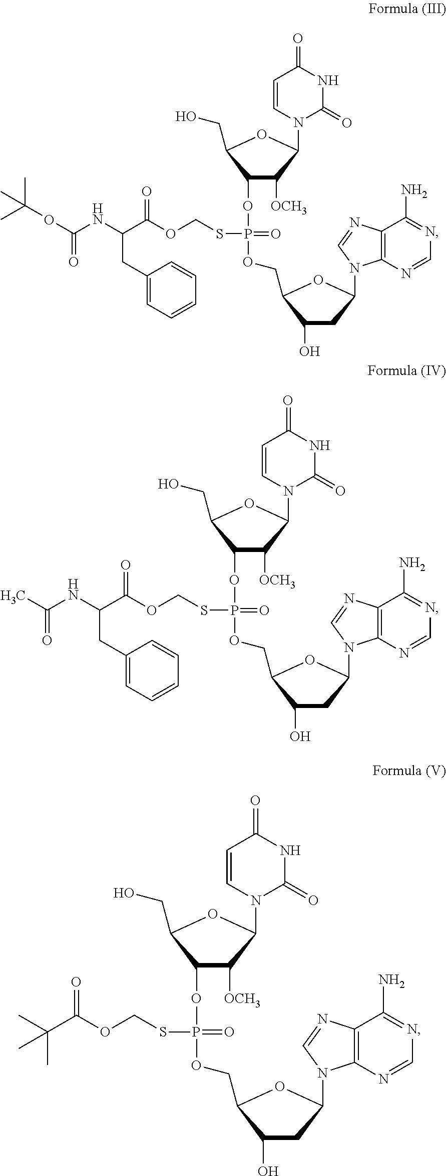 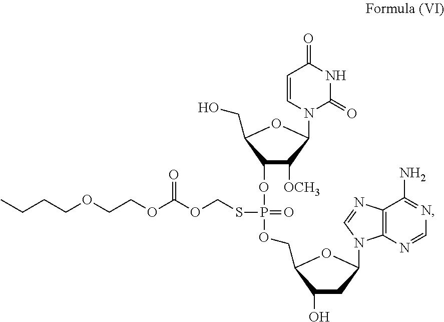
or a pharmaceutically acceptable salt thereof.

8. The method of claim 1, wherein the subject is administered a composition comprising a mixture of compounds of Formula (I).

9-18. (canceled)

19. The method of claim 1, wherein the method is a method of treating RSV infection.

20-24. (canceled)

25. The method of claim 1, wherein the method is a method of treating influenza.

26. (canceled)

27. The method of claim 1 wherein the method is a method of treating parainfluenza.

28. The method of claim 1 wherein the method is a method of treating rhinovirus or adenovirus.

29. The method of claim 1, wherein the subject is suffering from bronchiolitis, pneumonia, or other respiratory illness or condition.

30-47. (canceled)

48. The method of claim 1, further comprising the administration of a therapeutically effective amount of an additional agent.

49-56. (canceled)

57. A method for preventing infection with respiratory syncytial virus (RSV), influenza, adenovirus or rhinovirus in a subject, the method comprising administering to the subject a compound of Formula (I), wherein the compound is selected from: 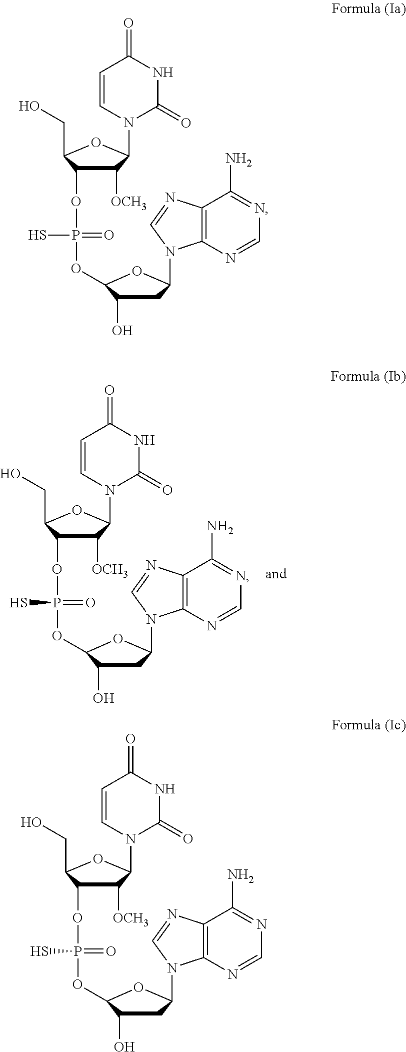
or a prodrug or pharmaceutically acceptable salt thereof.

58-59. (canceled)

60. The method of claim 57, wherein the prodrug of Formula (I) is a compound of Formula (P) 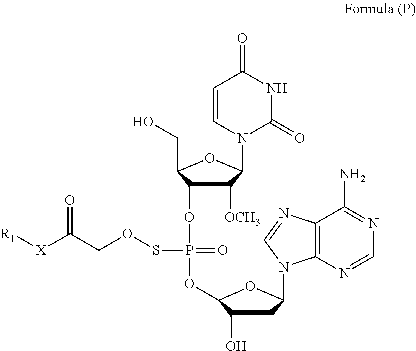
or a pharmaceutically acceptable salt thereof,
wherein,
X is a bond, O, NH, or S; and
R¹ is optionally substituted alkyl or heteroalkyl.

61. The method of claim 57, wherein the prodrug of Formula (I) is a compound of Formula (II), wherein the compound is selected from: 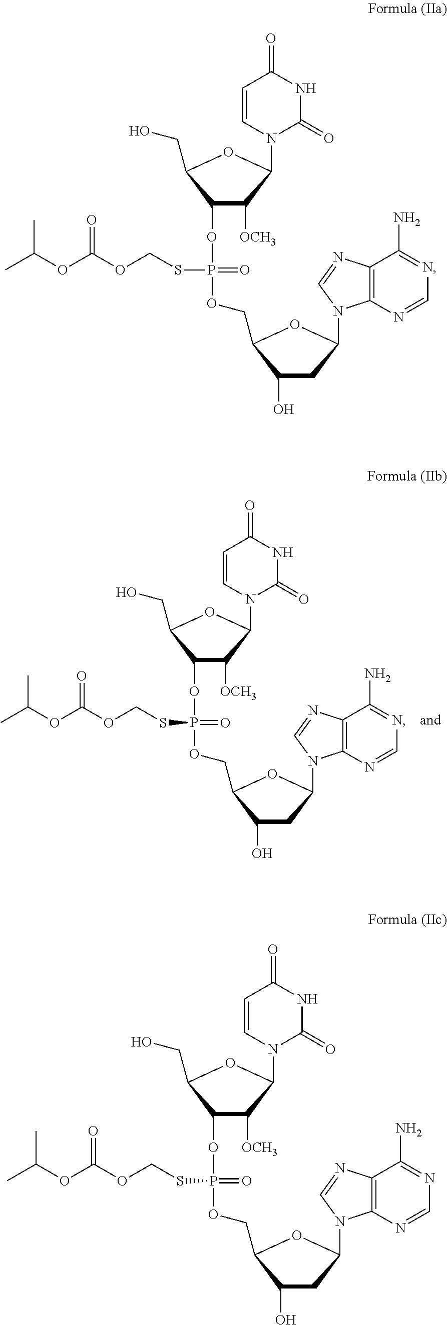
or a pharmaceutically acceptable salt thereof.

62. (canceled)

63. The method of claim 57, wherein the subject is administered a composition comprising a mixture of compounds of Formula (I).

64-73. (canceled)

74. The method of claim 57, wherein the method is a method of preventing RSV infection.

75-78. (canceled)

79. The method of claim 57, wherein the method is a method of preventing influenza.

80. (canceled)

81. The method of claim 57, wherein the method is a method of preventing parainfluenza.

82. The method of claim 57, wherein the method is a method of preventing rhinovirus or adenovirus infection.

83. The method of claim 57, wherein the subject is suffering from bronchiolitis, pneumonia, or other respiratory illness or condition.

84-101. (canceled)

102. The method of claim 57, further comprising the administration of a therapeutically effective amount of an additional agent.

103-110. (canceled)
